(19) United States
(12) Patent Application Publication
(10) Pub. No.: US 2010/0143381 A1
(43) Pub. Date: Jun. 10, 2010

(54) LYSOPHOSPHATIDIC ACID RECEPTOR TARGETING FOR LUNG DISEASE

(76) Inventors: Andrew Tager, Cambridge, MA (US); Andrew D. Luster, Wellesley, MA (US)

Correspondence Address: Carroll, Peter G.;Medlen & Carroll, LLP, 101 Howard Street, Suite 350, San Francisco, CA, 94105, US

(21) Appl. No.: 12/450,051

(22) PCT Filed: Mar. 11, 2008
(86) PCT No.: PCT/US2008/003167

§ 371 Date: Jan. 13, 2010

Related U.S. Application Data
(60) Provisional application No. 60/906,398, filed on Mar. 12, 2007.

(51) Int. Cl. A61K 39/395; A61K 31/7052; G01N 33/53; C12Q 1/68; A61K 38/16; A61P 11/00

(52) U.S. Cl. 424/172.1; 514/44 R; 435/7.1; 435/6; 514/12

(57) ABSTRACT

The present invention contemplates that lysophosphatidic acid (LPA) may be induced by lung injury and may be responsible for aberrant wound-healing responses. For example, in a bleomycin model of pulmonary fibrosis LPAi deficient mice are protected from pulmonary fibrosis and mortality. Specifically, chemotactic-induced fibroblast responses, lung fibroblast accumulation, and vascular permeability increases were all attenuated. In contrast, however, bleomycin-induced leukocyte recruitment was preserved. These results demonstrate that LPAi activity may link pulmonary fibrosis with lung injury by mediating fibroblast recruitment and vascular leak. The present invention therefore represents LPAi as a new target to treat lung diseases including, but not limited to, fibrosis, idiopathic pulmonary fibrosis, and acute respiratory distress syndrome.

### STATEMENT OF GOVERNMENT INTEREST

The present invention was funded by National Institutes of Health grants R01-CA89228, R01-CA095042, R01-MH51699, K02-MH01723, R01-NS048478, and R01-CA69212. Therefore, the United States government has certain rights to this invention.

### FIELD OF INVENTION

The present invention is related to the treatment of fibrotic diseases. For example, a fibrotic disease may include, but is not limited to, a pulmonary disease characterized by the generation of lysophosphatidic acid (LPA). The present invention contemplates methods and compositions related to the effective treatment of fibrotic lung diseases by administering inhibitory compounds directed to an LPA receptor. For example, one such receptor comprises LPA₁.

### BACKGROUND

Tissue injury initiates a complex series of host wound-healing responses. If successful, these responses restore normal tissue structure and function. If not successful, these responses can lead to tissue fibrosis and loss of function. In the lung, aberrant wound-healing responses to injury are thought to contribute to the pathogenesis of idiopathic pulmonary fibrosis (IPF). Selman et al., “Idiopathic pulmonary fibrosis: prevailing and evolving hypotheses about its pathogenesis and implications for therapy” Ann Intern Med 134:136-151 (2001).

IPF and other fibrotic lung diseases are associated with high morbidity and mortality, and are generally refractory to currently available pharmacological therapies. Better identification of the mediators linking lung injury and pulmonary fibrosis is needed to recognize new therapeutic targets for these important diseases.

### SUMMARY

The present invention is related to the treatment of fibrotic diseases. For example, a fibrotic disease may include, but is not limited to, a pulmonary disease characterized by the generation of lysophosphatidic acid (LPA). The present invention contemplates methods and compositions related to the effective treatment of fibrotic lung diseases by administering inhibitory compounds directed to an LPA receptor. For example, one such receptor comprises LPA₁.

In one embodiment, the present invention contemplates a method, comprising: a) providing: i) a subject at risk for an injury, wherein said injury is likely to result in a fibrosis; ii) a composition comprising an inhibitory compound having affinity for at least a fragment of a lysophosphatidic acid receptor; and b) administering said composition to said subject before said injury, under conditions such that said fibrosis is prevented or reduced. In one embodiment, the injury comprises a pulmonary injury. In one embodiment, the pulmonary injury is selected from the group consisting of toxin inhalation injury, surgical procedure injury, infection, and accidental injury. In one embodiment, the fibrosis comprises symptoms selected from the group consisting of fibroblast migration and vascular leak. In one embodiment, the composition further comprises at least one additional drug. In one embodiment, the drug is selected from the group consisting of antiproliferative drugs, anticoagulant drugs, antithrombotic drugs, and antiplatelet drugs. In one embodiment, the administering is selected from the group consisting of topical, oral, parenteral, pulmonary, anal, vaginal, ocular, and intranasal.

In one embodiment, the present invention contemplates a method, comprising: a) providing: i) a subject comprising a progressive injury, wherein said injury promotes fibrosis; ii) a composition comprising an inhibitory compound having affinity for at least a fragment of a lysophosphatidic acid receptor; and b) administering said composition to said subject before said injury, under conditions such that said fibrosis is prevented or reduced. In one embodiment, the injury comprises a pulmonary injury. In one embodiment, the progressive injury results in an increase in said fibrosis. In one embodiment, the pulmonary injury is selected from the group consisting of toxin inhalation injury, surgical procedure injury, infection, and accidental injury. In one embodiment, the fibrosis comprises symptoms selected from the group consisting of fibroblast migration and vascular leak. In one embodiment, the composition further comprises at least one additional drug. In one embodiment, the drug is selected from the group consisting of antiproliferative drugs, anticoagulant drugs, antithrombotic drugs, and antiplatelet drugs. In one embodiment, the administering is selected from the group consisting of topical, oral, parenteral, pulmonary, anal, vaginal, ocular, and intranasal.

In one embodiment, the present invention contemplates a method, comprising: a) providing: i) a subject comprising an injury, wherein said injury resulted in a fibrosis; ii) a composition comprising an inhibitory compound having affinity for at least a fragment of a lysophosphatidic acid receptor; and b) administering said composition to said subject after said injury, under conditions such that said fibrosis is reduced. In one embodiment, the injury comprises a pulmonary injury. In one embodiment, the pulmonary injury is selected from the group consisting of toxin inhalation injury, surgical procedure injury, infection, and accidental injury. In one embodiment, the fibrosis comprises symptoms selected from the group consisting of fibroblast migration and vascular leak. In one embodiment, the composition further comprises at least one additional drug. In one embodiment, the drug is selected from the group consisting of antiproliferative drugs, anticoagulant drugs, antithrombotic drugs, and antiplatelet drugs. In one embodiment, the administering is selected from the group consisting of topical, oral, parenteral, pulmonary, anal, vaginal, ocular, and intranasal.

In one embodiment, the present invention contemplates a method, comprising: a) providing; i) an isolated lysophosphatidic acid receptor, wherein said receptor is derived from a fibroblast; and ii) a test compound capable of an interaction with said receptor; b) contacting said receptor with said test compound; and c) detecting said interaction of said receptor with said test compound. In one embodiment, the fibroblast is derived from a pulmonary tissue. In one embodiment, the test compound comprises a protein. In one embodiment, the test compound comprises a small organic molecule. In one embodiment, the protein comprises a fusion peptide. In one embodiment, the test compound comprises a nucleic acid. In one embodiment, the protein comprises an antibody. In one embodiment, the protein comprises a peptide. In one embodiment, the receptor comprises and LPA receptor. In one embodiment, the LPA receptor is an LPA₁ receptor.

In one embodiment, the present invention contemplates a kit comprising: a) a nucleic acid capable of hybridizing to at least a portion of an LPA₁ receptor deoxyribonucleic acid (DNA) sequence; b) at least one sample comprising said LPA₁ receptor DNA sequence; and c) a set of instructions for using said nucleic acid to detect said LPA₁ receptor DNA sequence. In one embodiment, said at least one sample comprises a patient sample. In one embodiment, the patient sample comprises lung tissue. In one embodiment, said at least one sample comprises a wild-type fibroblast cell culture sample. In one embodiment, the DNA sequence comprises an LPA₁ coding region. In one embodiment, the nucleic acid comprises a primer. In one embodiment, the kit further comprises at least one polymerase enzyme. In one embodiment, the instructions further provide for using said DNA sequence detection to diagnose fibrosis. In one embodiment, the fibrosis is pulmonary fibrosis. In one embodiment, said instructions further diagnose fibrosis by comparing said patient sample detected DNA sequence to said cell culture detected DNA sequence.

In one embodiment, the present invention contemplates a kit comprising: a) a nucleic acid capable of hybridizing to at least a portion of an LPA₁ receptor messenger ribonucleic acid (mRNA) sequence; b) at least one sample comprising said LPA₁ receptor mRNA sequence; and c) a set of instructions for using said nucleic acid to detect said LPA₁ receptor mRNA sequence. In one embodiment, the nucleic acid sequence comprises a primer. In one embodiment, the kit further comprises at least one polymerase. In one embodiment, said at least one sample comprises a patient sample. In one embodiment, the patient sample comprises lung tissue. In one embodiment, said at least one sample comprises a wild-type fibroblast cell culture sample. In one embodiment, the mRNA sequence comprises an LPA₁ coding region. In one embodiment, the instructions further provide for using said mRNA sequence detection to diagnose fibrosis. In one embodiment, the fibrosis is pulmonary fibrosis. In one embodiment, said instructions further diagnose fibrosis by comparing said patient sample detected mRNA sequence to said cell culture detected mRNA sequence.

In one embodiment, the present invention contemplates a kit comprising: a) at least one antibody capable of binding to an LPA, receptor protein; b) at least one sample comprising said LPA₁ receptor protein; and c) a set of instructions for using said at least one antibody to detect said LPA₁ receptor protein. In one embodiment, the at least one antibody comprises a first labeled antibody. In one embodiment, the at least one antibody comprises a second labeled antibody. In one embodiment, said at least one sample comprises a patient sample. In one embodiment, the patient sample comprises lung tissue. In one embodiment, said at least one sample comprises a wild-type fibroblast cell culture sample. In one embodiment, said first antibody comprises a high affinity for an LPA₁ receptor epitope. In one embodiment, said second antibody comprises a high affinity for said first antibody. In one embodiment, the instructions further provide for using said LPA₁ receptor protein detection to diagnose fibrosis. In one embodiment, the fibrosis is pulmonary fibrosis. In one embodiment, said instructions further diagnose fibrosis by comparing said patient sample detected LPA₁ receptor protein to said cell culture detected LPA₁ receptor protein.

In one embodiment, the present invention contemplates a kit comprising: a) an LPA₁ receptor inhibitor; and b) a pharmaceutically acceptable carrier capable of administering said inhibitor to a subject. In one embodiment, the inhibitor comprises a nucleic acid capable of hybridizing to at least a portion of an LPA₁ receptor coding region. In one embodiment, the inhibitor comprises an antibody capable of binding to an LPA₁ receptor protein. In one embodiment, the inhibitor comprises a small organic molecule capable of binding to an LPA₁ receptor protein. In one embodiment, the inhibitor comprises a protein capable of binding to an LPA₁ receptor protein. In one embodiment, the kit further comprises a set of instructions for administering said receptor inhibitor to said subject.

### Definitions

The term “fibrosis” as used herein, refers to any medical condition marked by increase of interstitial fibrous tissue. For example, “pulmonary fibrosis” is characterized by a scarring or thickening of the lungs.

The term “inhibitory compound” as used herein, refers to any compound capable of interacting with (i.e., for example, attaching, binding etc) to a binding partner (i.e., for example, an LPA₁ receptor) under conditions such that the binding partner becomes unresponsive to its natural ligands. Inhibitory compounds may include, but are not limited to, small organic molecules, antibodies, and proteins/peptides.

The term “lysophosphatidic acid receptor” as used herein, refers to any protein capable of binding lysophosphatidic acid (LPA). For example, an LPA receptor may reside in the cell membrane and respond to circulating levels of LPA in order to mediate various physiological responses. The type of response depends upon LPA receptor subtype (i.e., for example, LPA₁, LPA₂, LPA₃, LPA₄, LPA₅).

The term “pulmonary injury” as used herein, refers to any effect on pulmonary tissue that impairs it functional or structural integrity. For example, injury may be a result of, but not limited to, inhalation of toxins, surgical procedures, or accident.

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc. Conditions related to wounds or sores which may be successfully treated according to the invention are skin diseases.

The term “fibroblast migration” as used herein, refers to any movement of a fibroblast in the direction of tissue injury. Such migration is usually stimulated by chemotactic factors (i.e., for example, lysophosphatidic acid) released by white blood cells.

The term “vascular leak” as used herein, refers to an increase in vascular permeability due to tissue injury. Such a condition may result in internal bleeding and blood coagulation, inflammation, and ultimately the development of fibrosis.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “medium” as used herein, refers to any material, or combination of materials, which serve as a carrier or vehicle for delivering of a drug to a treatment point (e.g., wound, surgical site etc.). For all practical purposes, therefore, the term “medium” is considered synonymous with the term “carrier”. It should be recognized by those having skill in the art that a medium comprises a carrier, wherein said carrier is attached to a drug or drug and said medium facilitates delivery of said carrier to a treatment point. Further, a carrier may comprise an attached drug wherein said carrier facilitates delivery of said drug to a treatment point. Preferably, a medium is selected from the group including, but not limited to, foams, gels (including, but not limited to, hydrogels), xerogels, microparticles (i.e., microspheres, liposomes, microcapsules etc.), bioadhesives, or liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of drugs over a period of time lasting approximately from 1 day to 6 months.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering” a drug or compound, as used herein, refers to any method of providing a drug or compound to a patient such that the drug or compound has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “antiplatelets” or “antiplatelet drug” as used herein, refers to any drug that prevents aggregation of platelets or fibrin formation (i.e., for example as a prior event to adhesion formation). For example, an antiplatelet drug comprises an inhibitor of glycoprotein IIb/IIIa (GPIIb/IIIa). Further a GPIIb/IIIa inhibitor includes, but is not limited to, xemilofiban, abciximab (ReoPro®) cromafiban, elarofiban, orbofiban, roxifiban, sibrafiban, RPR 109891, tirofiban (Aggrastat®), eptifibatide (Integrilin®), UR-4033, UR-3216 or UR-2922.

The term, “antithrombins” or “antithrombin drug” as used herein, refers to any drug that inhibits or reduces thrombi formation and include, but are not limited to, bivalirudin, ximelagatran, hirudin, hirulog, argatroban, inogatran, efegatran, or thrombomodulin.

The term, “anticoagulants” or “anticoagulant drug” as used herein, refers to any drug that inhibits the blood coagulation cascade. A typical anticoagulant comprises heparin, including but not limited to, low molecular weight heparin (LMWH) or unfractionated heparin (UFH). Other anticoagulants include, but are not limited to, tinzaparin, certoparin, parnaparin, nadroparin, ardeparin, enoxaparin, reviparin or dalteparin. Specific inhibitors of the blood coagulation cascade include, but are not limited to, Factor Xa (FXa) inhibitors (i.e., for example, fondaparinux), Factor IXa (FIXa) inhibitors, Factor XIIIa (FXIIIa) inhibitors, and Factor VIIa (FVIIa) inhibitors.

The term “patient”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “effective amount” as used herein, refers to a particular amount of a pharmaceutical composition comprising a therapeutic agent (i.e., for example, an LPA₁ receptor inhibitor) that achieves a clinically beneficial result.

The term “derived from” as used herein, refers to the source of a compound or sequence. In one respect, a compound or sequence may be derived from an organism or particular species. In another respect, a compound or sequence may be derived from a larger complex or sequence.

The term “test compound” as used herein, refers to any compound or molecule considered a candidate as an inhibitory compound.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabelled A) in a reaction containing labeled “A” and the antibody will reduce the amount of labeled A bound to the antibody.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (-) (i.e., “negative”) is sometimes used in reference to the antisense strand, with the designation (+) sometimes used in reference to the sense (i.e., “positive”) strand.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample suspected of containing nucleic acid encoding a LPA receptor protein may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence (i.e., for example, SEQ ID NO:1) or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.). Included within this definition are alterations to the genomic DNA sequence which encodes LPA₁ (i.e., for example, by alterations in the pattern of restriction enzyme fragments capable of hybridizing to SEQ ID NO:1 (RFLP analysis), the inability of a selected fragment to hybridize under high stringency conditions to a sample of genomic DNA (e.g., using allele-specific oligonucleotide probes), and improper or unexpected hybridization, such as hybridization to a locus other than the normal chromosomal locus for the LPA₁ gene (e.g., using fluorescent in situ hybridization (FISH)).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, for example, naturally occurring LPA₁.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions. For example, LPA₁ receptor biological activity may be determined, for example, by restoration of wild-type growth in cells lacking an LPA₁ receptor (i.e., for example, LPA₁ receptor protein null cells and/or “knock out” cells). Cells lacking LPA₁ receptors may be produced by many methods (i.e., for example, point mutation and frame-shift mutation). Complementation is achieved by transfecting cells which lack LPA₁ receptors with an expression vector which expresses LPA₁ receptor protein, a derivative thereof, or a portion thereof.

The term “immunologically active” defines the capability of a natural, recombinant or synthetic peptide (i.e., for example, a collagen-like family protein), or any oligopeptide thereof, to induce a specific immune response in appropriate animals or cells and/or to bind with specific antibodies.

The term “antigenic determinant” as used herein refers to that portion of a molecule that is recognized by a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “immunogen,” “antigen,” “immunogenic” and “antigenic” refer to any substance capable of generating antibodies when introduced into an animal. By definition, an immunogen must contain at least one epitope (the specific biochemical unit capable of causing an immune response), and generally contains many more. Proteins are most frequently used as immunogens, but lipid and nucleic acid moieties complexed with proteins may also act as immunogens. The latter complexes are often useful when smaller molecules with few epitopes do not stimulate a satisfactory immune response by themselves.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” of the LPA₁ gene of SEQ ID NO: 1 is defined herein as an oligonucleotide sequence which exhibits greater than or equal to 50% identity to the sequence of SEQ ID NO: 1 when sequences having a length of 100 by or larger are compared. Alternatively, a homolog of SEQ ID NO: 1 is defined as an oligonucleotide sequence which encodes a biologically active LPA₁ receptor amino acid sequence. For example, an LPA₁ homolog may comprise a portion of an oligonucleotide sequence encoding an LPA₁ receptor amino acid sequence.

Low stringency conditions comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent {50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ to about 20° C. to 25° C. below Tₘ. A “stringent hybridization” can be used to identify or detect identical polynucleotide sequences or to identify or detect similar or related polynucleotide sequences. For example, when fragments of SEQ ID NO:2 are employed in hybridization reactions under stringent conditions the hybridization of fragments of SEQ ID NO:2 which contain unique sequences (i.e., regions which are either non-homologous to or which contain less than about 50% homology or complementarity with SEQ ID NOs:2) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40.

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane: The immobilized DNA is then probed with a labeled oligoribonuclotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample suspected of containing nucleic acid encoding a collagen-like family protein may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “binding” as used herein, refers to any interaction between an infection control composition and a surface. Such as surface is defined as a “binding surface”. Binding may be reversible or irreversible. Such binding may be, but is not limited to, non-covalent binding, covalent bonding, ionic bonding, Van de Waal forces or friction, and the like. An infection control composition is bound to a surface if it is impregnated, incorporated, coated, in suspension with, in solution with, mixed with, etc.

### BRIEF DESCRIPTION OF THE FIGURES

FIG. 1 presents exemplary data showing that bleomycin-induced fibroblast chemoattractant activity can be generated in lung airspaces that co-purifies with albumin:

FIG. 1a shows fibroblast chemotactic activity induced by BAL samples recovered from mice on Day 0, Day 5, Day 10 and Day 14 post-bleomycin-induced injury. Data are from one of two independent experiments with similar results, and are presented as mean chemotactic index (cells counted in duplicate wells moving in response to BAL relative to cells moving in response to media control)+SEM. BAL from n=4 mice at each time point, *P<0.05 indicates significant chemotaxis induced by Day 5 (D5), Day 10 (D10) or Day (D14) BAL samples in comparison to Day 0 (D0) BAL samples.

FIG. 1b shows the sensitivity of BAL-induced fibroblast chemotaxis to pertussis toxin (PTX). PTX-pretreatment of fibroblasts inhibited chemotaxis induced by BAL from mice on D5 post-bleomycin administration, but not chemotaxis induced by PDGF. D5 BAL from n=8 mice, P<0.0001 indicates significant chemotaxis induced by D5 BAL of PTX-treated fibroblasts in comparison to. untreated fibroblasts.

FIG. 1c shows the fibroblast chemotactic indices of filtrates and retentates produced by size exclusion centrifugation of BAL across filters with molecular exclusion sizes of 30, 50, and 100 kDa. Chemotactic activity was restricted to the retentates produced by the 30 and 50 kDa filters, but was present in both the retentate and the filtrate produced by the 100 kDa filter.

FIG. 1d presents results from heparin affinity chromatography of BAL fibroblast chemoattractants. BAL was loaded onto a 5 ml HiTrap Heparin HP column, and eluted with a linear gradient of 0 to 2M NaCl. Dashed line indicates eluate conductivity.

FIG. 1e presents results from fibroblast chemotactic indices of heparin affinity fractions. Chemotactic activity was present in the flow through (fraction 1), and the fractions eluted with the lowest concentrations of NaCl (fractions 6 to 8), which contained the BAL proteins with the weakest heparin binding affinities.

FIG. 1f presents hydrophobic interaction chromatography of BAL fibroblast chemoattractants. BAL was dialyzed against 1.7 M ammonium sulfate, loaded onto a 1 ml RESOURCE PHE column, and eluted with a linear gradient of 1.7 to 0.0 M ammonium sulfate. Dashed line indicates eluate conductivity.

FIG. 1g presents fibroblast chemotactic indices of hydrophobic interaction fractions. Chemotactic activity was present in the fractions eluted with the lowest ammonium sulfate concentration (fractions 18-23), which contained the BAL proteins with the strongest hydrophobic interactions.

FIG. 1h presents an exemplary dose response curve for LPA (10⁻¹²-10⁻⁴ M) for fibroblast chemotactic indices.

FIG. 1i shows the sensitivity of LPA-induced fibroblast chemotaxis to pertussis toxin (PTX). PTX-pretreatment of fibroblasts inhibited chemotaxis induced by several concentrations of LPA (10⁻⁹-10⁻⁷ M), but not chemotaxis induced by PDGF concentrations(10⁻⁹-10⁻⁷ ).

FIG. 1j shows LPA concentrations in BAL samples following bleomycin-induced lung injury. LPA concentrations (y.axis) were determined by electrospray ionization mass spectrometry in BAL samples from unchallenged mice (Day 0) and BAL samples collected from mice 5, 7, 10 and 14 days post-bleomycin challenge (x axis). N=4 mice per time point, *P<0.01 indicates significance when comparing LPA concentrations from BAL samples at 5 and 10 days post-bleomycin in comparison to Day 0, and **P<0.05 indicates significance when comparing LPA concentrations from BAL samples at 7 and 14 days post-bleomycin in comparison to Day 0.

FIG. 1k shows lung fibroblast LPA receptor expression. Quantitative polymerase chain reaction of mRNA extracted from cultures of primary lung fibroblasts demonstrated high levels of LPA₁ mRNA. Data are presented as copies of LPA₁ receptor mRNA relative to copies of GAPDH mRNA+SEM. mRNA was isolated from n=3 fibroblast cultures, prepared from sets of two C57BI/6 mice each.

FIG. 2 presents exemplary data from SDS-PAGE electrophoresis of the protein characterization experiments in FIG. 1:

FIG. 2a presents the SDS-PAGE banding patterns of BAL size exclusion centrifugation fractions following a Comassie stain.

FIG. 2b presents the SDS-PAGE banding patterns of BAL heparin affinity fractions.

FIG. 2c presents the SDS-PAGE banding patterns of BAL hydrophobic interaction fractions.

FIG. 3 presents exemplary data showing PECAM-1 expression of mouse primary cardiac endothelial cells. Endothelial cells isolated from heart tissues of C57BL/6 mice were stained with anti-PECAM-1 antibody (grey shaded histogram) or isotype control (open histogram). 93% of endothelial cells were PECAM-1 positive.

FIG. 4 presents illustrative data showing that LPA₁-deficient (LPA₁ KO) mice are protected from bleomycin-induced fibrosis and mortality.

FIG. 4a shows parenchymal abnormalities in wild type mice 14 days post-bleomycin challenge. Cells stained with hemotoxylin and eosin at 100× magnification.

FIG. 4b shows parenchymal abnormalities in LPA₁^{-/-} mice 14 days post-bleomycin challenge. Cells stained with hemotoxylin and eosin at 100× magnification.

FIG. 4c shows collagen accumulation in wild type mice 14 days post-bleomycin challenge. Cells are stained with trichrome at 400× magnification.

FIG. 4d shows collagen accumulation in LPA1−/− mice 14 days post-bleomycin challenge. Cells are stained with trichrome at 400× magnification.

FIG. 4e presents exemplary data of a biochemical analysis of bleomycin-induced fibrosis. Hydroxyproline content was measured in the lungs of wild type (WT) and LPA₁^{-/-} mice at baseline and on Day 14 following bleomycin administration (D0=untreated; n=5 mice/group) D14=fourteen days post-bleomycin administration; n=8 mice/group). Data presented are from one of two independent experiments with similar results, and are expressed as mean hydroxyproline content per lung set+SEM. Test for interaction between genotype and bleomycin treatment by two-way analysis of variance for independent samples was significant at P=0.0073.

FIG. 4f presents exemplary data showing bleomycin-induced lung collagen expression. QPCR analysis of expression of the α2 chain of procollagen type I in mRNA isolated from the lungs of WT and LPA₁ KO (LPA₁^{-/-}) mice at baseline, and on Day 5 (D5) and Day 14 (D14) following bleomycin (D0 untreated, n=3 mice/group; D5 and D14 post-bleomycin, n>5 mice/group; *P<0.05 procollagen expression induced at D14 post-bleomycin in WT vs. LPA₁ KO lungs). Data are expressed as mean copies of procollagen mRNA relative to copies of α2 microglobulin mRNA±SEM.

FIG. 4g presents exemplary data of bleomycin-induced mortality. WT and LPA₁^{-/-} mice were followed for survival for 21 days after challenge with 3 units/kg of bleomycin. n=10 mice/group. Significant difference by log rank test: P=0.0115 LPA₁^{-/-} vs. wild type (WT) survival.

FIG. 5 presents exemplary data showing that fibroblast chemotaxis induced by bleomycin injury is diminished in LPA₁^{-/-} mice.

FIG. 5a shows that LPA-mediated chemotaxis of lung fibroblasts is at least partially mediated by LPA₁ receptors. Compared with chemotaxis of WT fibroblasts, the chemotaxis of LPA₁^{-/-} fibroblasts induced by various concentrations of LPA (10⁻⁹-10⁻⁷ M) is reduced (* P<0.01), but the chemotaxis of LPA₁^{-/-} fibroblasts induced by PDGF (10⁻⁹ M) is not affected.

FIG. 5b presents exemplary data showing that C57BL fibroblast chemotaxis induced by a BAL sample collected on Day 5 after bleomycin administration is inhibited by an LPA₁ antagonist (i.e., for example, 1 μM Ki16425) Chemotaxis induced by 10⁻⁸ or 10⁻⁷ M PDGF was not affected. n=4, *P=0.0033.

FIG. 5c presents exemplary data showing fibroblast chemotaxis induced by BAL samples collected on Day 5 (D5), Day 10 (D10), and Day 14 (D14) after bleomycin administration is reduced in LPA₁^{-/-} fibroblasts (LPA₁ KO). Compared with chemotaxis of WT fibroblasts, LPA₁ KO fibroblasts demonstrated reduced chemotaxis to BAL fluid from mice on D5, D10, or D14 post-bleomycin injury. Chemotaxis induced by 10⁻⁸ or 10⁻⁷ M PDGF was not affected. n=4 C57BL/6 mice per time point, *P<0.005.

FIG. 5d shows accumulation of FSP1-staining fibroblasts following bleomycin challenge in WT and LPA₁^{-/-} mice. Top Panel: Lungs of WT and LPA₁^{-/-} mice before bleomycin challenge. Bottom Panel: Lungs of WT and LPA₁^{-/-} mice on Day 14 after bleomycin challenge. Cells were stained with anti-FSP1 antibody/peroxidase (Magnification 400×).

FIG. 5e shows exemplary data from 10 randomly selected lung sections quantitating FSP1-staining cells in WT and LPA₁^{-/-} mice using image analysis software. D0: Pre-Bleomycin challenge. D14: Day 14 days after a bleomycin challenge. n=3 mice per group, all groups. Data are expressed as mean FSP1 staining area+SEM; Day 14 comparison of LPA₁^{-/-} D14 and WT D14 was statistically significant (P=0.0017).

FIG. 5f shows generation of a Day 5 bleomycin-induced BAL sample fibroblast chemotactic activity is independent of LPA₁ receptors. BAL samples collected from WT and LPA₁^{-/-} bleomycin challenged mice were tested in vitro using WT C57BL/6 fibroblasts. n=4 mice, each genotype.

FIG. 5g presents exemplary data showing that proliferation of lung fibroblasts induced by Day 5 (D5) or Day 14 (D14) post-bleomycin BAL sample is independent of LPA₁ receptors. N=3 C57BL/6 mice. Data are presented as mean proliferative index (i.e., counts per minute (CPM) incorporated into cells proliferating in response to BAL or PDGF counted in triplicate wells relative to CPM incorporated into cells proliferating in media control)+SEM.

FIG. 5h presents exemplary data showing that fibroblast TGF-β-induced gene expression is not dependent on LPA₁ receptors. QPCR analysis of mRNA expression of procollagen type I α₁ (Col I) , fibronectin (FN), and α-smooth muscle actin (αSMA) induced in WT and LPA₁ KO lung fibroblasts by 24 hr exposure to TGF-β (10 μg/ml). Data are expressed as fold induction (mean copies of each gene relative to copies of α2 microglobulin mRNA in TGF-β-exposed cells divided by mean copies in non-exposed cells)±SEM, and are from n≧3 fibroblasts cultures per genotype per condition (media with or without TGF-β).

FIG. 6 presents exemplary data showing that vascular leak induced by bleomycin injury is diminished in LPA₁^{-/-} mice:

FIG. 6a presents exemplary data showing expression patterns of various types of endothelial cell LPA receptors. QPCR of mRNA isolated from primary mouse lung endothelial cells demonstrated a high expression of the LPA₁ receptor. Data are presented as copies of receptor mRNA relative to copies of GAPDH mRNA.

mRNA.

FIG. 6b presents exemplary data showing lung vascular leak induced by bleomycin injury assessed by extravasation of Evans blue dye. Gross appearance of lungs from representative wild type mice (left) and LPA₁ KO mice (right) seven days after a bleomycin challenge.

FIG. 6c presents Evans blue dye indices (i.e., for example, the total amount of lung Evans blue dye/plasma concentration of Evans blue dye) to quantitate vascular leakage (i.e., for example, an increase in vascular permeability). Wild type (WT) and LPA₁^{-/-} mice were compared before bleomycin challenge (D0) and on Day 7 (D7) after bleomycin challenge. WT mice: n=5 at D0 and D7. LPA₁^{-/-} mice: n=4 at D0, and n=3 at D7. Data presented are from one of three independent experiments with similar results, and are expressed as mean Evans blue index+SEM. * P=0.025, LPA₁^{-/-} versus WT D7.

FIG. 6d presents exemplary data showing lung vascular leak induced by bleomycin injury as assessed by BAL sample total protein concentration. The increase in BAL sample protein concentration in WT mice post-bleomycin challenge was smaller in LPA₁ KO mice (BAL samples from n≧4 WT and LPA₁ KO mice at each time point). Data presented are from one of two independent experiments with similar results, and are expressed as mean BAL total protein concentration±SEM. (*P<0.05, LPA₁ KO vs. WT at D3, D5, D7 and D14 after bleomycin administration).

FIG. 7 presents exemplary data showing preservation of leukocyte recruitment and activation induced by bleomycin injury in LPA₁^{-/-} mice.

FIG. 7a presents exemplary data showing leukocyte LPA receptor expression using QPCR of mRNA isolated from myeloid cells (alveolar macrophages and neutrophils). Data are presented as copies of receptor mRNA relative to copies of GAPDH mRNA.

FIG. 7b presents exemplary data showing leukocyte LPA receptor expression using QPCR of mRNA isolated from lymphocytes (CD4+ and CD8+ T cells). Data are presented as copies of receptor mRNA relative to copies of GAPDH mRNA.

FIG. 7c show the total cell count in bleomycin-induced BAL samples. Cells were counted using a hemocytometer as recovered in BAL samples from wild type (WT) and LPA₁^{-/-} mice on Day 1, Day 3, Day 5, Day 7 and Day 14 after a bleomycin challenge. Data presented are from one of two independent experiments, and are expressed as mean cell numbers+SEM. n=4 mice.

FIG. 7d show the number of macrophages recovered in BAL samples from WT and LPA₁^{-/-} mice on Day 1, Day 3, Day 5, Day 7, and Day 14 following bleomycin challenge. Determinations were made by multiplying total BAL cells by subset percentages from cytospin preparations of BAL samples stained with Hema 3 stain. n=4 mice. *P<0.05, BAL macrophages at D14 post-bleomycin in WT vs. LPA₁ KO mice.

FIG. 7e show the number of neutrophils recovered in BAL samples from WT and LPA₁^{-/-} mice on Day 1, Day 3, Day 5, Day 7, and Day 14 following bleomycin challenge. Determination were made by multiplying total BAL cells by subset percentages from cytospin preparations of BAL samples stained with Hema 3 stain. n=4 mice.

FIG. 7f show the number of T cells (CD3⁺) recovered in BAL samples from WT and LPA₁^{-/-} mice on Day 3, Day 5, Day 7 and Day 14 following bleomycin challenge. Determinations were made by multiplying total BAL cells by subset percentages using flow cytometry. n=4 mice.

FIG. 7g shows the number of CD4⁺ T cells (CD3⁺, CD4⁺) recovered in BAL samples from WT and LPA₁^{-/-} mice on Day 3, Day 5, Day 7, and Day 14 following bleomycin challenge. Determinations were made by multiplying total BAL cells by subset percentages using flow cytometry. n=4 mice.

FIG. 7h shows the number of CD8⁺ T cells (CD3⁺,CD8⁺) recovered in BAL samples from WT and LPA₁^{-/-} mice on Day 3, Day 5, Day 7, and Day 14 following bleomycin challenge. Determinations were made by multiplying total BAL cells by subset percentages using flow cytometry. n=4 mice. differences: *P<0.05, BAL CD8⁺ cells at D5 and D14 post-bleomycin in WT vs. LPA₁ KO mice.

FIG. 7i shows BAL T cell functional phenotype and activation status. Percentages of CD4⁺ and CD8⁺ T cells recovered in BAL from WT and LPA₁ KO mice on Day 5 following bleomycin challenge that were CD69⁺ and CD44⁺ were determined by flow cytometry. Data presented as mean percentages positive±SEM.

FIG. 8 presents exemplary data showing LPA and LPA1 receptor contributions to fibroblast chemoattractant activity in BAL of human IPF patients.

FIG. 8a shows procollagen type Iα1 (Col I) and CD14 receptor expression determined by QPCR techniques.

FIG. 8b shows LPA receptor expression of fibroblasts grown from BAL samples obtained from a human IPF patient.

FIG. 8c shows LPA concentrations in BAL samples obtained from humans. BAL LPA levels were compared between seven (7) IPF patients and three (3) healthy control subjects: *P<0.05. Concentrations of LPA were determined by electrospray ionization mass spectrometry in BAL from IPF patients (n=9) and normal controls (n=7). *P=0.029 comparing LPA concentration of patients vs. normals.

FIG. 8d shows that IPF BAL samples induce fibroblast chemotaxis that is inhibited by an LPA₁ antagonist. Compared with the chemotaxis of untreated cells, HFL1 cells treated with 1 μM Ki16425 demonstrated reduced chemotaxis to BAL samples from IPF patients. #P<0.0005, untreated vs. treated cells. BAL samples from human IPF patients induced significantly greater chemotaxis of human fetal lung fibroblasts (HFL1 cells) than BAL samples from controls. IPF patients (n=7); healthy controls (n=3). *P<0.05, IPF vs. controls

FIG. 9 presents exemplary data showing effects of albumin on LPA-induced chemotaxis.

FIG. 9a shows that methanol-extracted fatty acid-free mouse serum albumin (MSA) by itself did not induce chemotaxis of primary lung fibroblasts, whereas nonextracted MSA alone did (*P<0.0001, non-extracted vs. extracted MSA).

FIG. 9b shows that chemotaxis of lung fibroblasts from C57Bl/6 mice induced by LPA is potentiated by 0.1% fatty acid-free BSA. *P=0.0058, LPA alone vs. LPA+ fatty acid free BSA.

FIG. 10 presents exemplary data showing that fibroblast chemotaxis induced by BAL samples collected on Day 5 after bleomycin administration is inhibited by an LPA₁ antagonist. Compared with the chemotaxis of untreated fibroblasts, C57BL/6 fibroblasts treated with 100 μM VPC12249 demonstrated reduced chemotaxis to BAL sample from C57Bl/6 mice D5 post-bleomycin injury (*P=0.011). VPC12249 did not affect fibroblast chemotaxis induced by PDGF (10⁻⁹ M).

FIG. 11 presents exemplary data showing lung fibroblast LPA receptor expression.

FIG. 11a shows lung fibroblast LPA receptor expression before a bleomycin challenge in WT mice and LPA1 KO mice and

FIG. 11b shows lung fibroblast LPA receptor expression on Day 14 after a bleomycin challenge in WT mice and LPA₁ KO mice. QPCR of mRNA was isolated from n=3 cultures prepared from mice of each genotype at each time point. Data are presented as copies of receptor mRNA relative to copies of GAPDH mRNA±SEM.

FIG. 12 presents exemplary data showing apoptosis in the lungs of WT and LPA₁ KO mice. TUNEL assays were performed on lung sections of wild type and LPA1 KO mice sacrificed before (D0) and after (D7 and D14) bleomycin challenge. TUNEL+ cells present in the lungs were quantified by a pathologist blinded to mouse genotype and treatment group, by grading 10 non-overlapping high-power fields for each section using a semiquantitative scoring system. Each field was evaluated for: i) quantity of TUNEL+ cells. Scoring scale: 0=no positive cells; 1=1-5% positive cells; 2=5-25% positive cells; and 3>25% positive cells;

and ii) intensity of staining. Scoring scale: 1=weak; 2=moderate; and 3=strong. Each field's quantity score was multiplied by its intensity score to give an integrated apoptosis score (range 0-9), and the means of the integrated scores for the 10 fields examined were calculated for each mouse. Absence of LPA₁ expression was associated with greater apoptosis at baseline, but reduced apoptosis following bleomycin challenge. (n=3 mice per genotype per time point, *P<0.05).

FIG. 13 presents exemplary data showing fibrocyte accumulation induced by bleomycin in WT and LPA₁ KO mice. Surface staining with anti-CD45 antibody and intracellular staining with anti-collagen I (Col I) antibody was performed on single cell suspensions generated from the lungs of WT and LPA₁ KO mice before bleomycin challenge and on Day 7 following bleomycin challenge. Cells costaining with both antibodies were identified by flow cytometry. CD45⁺ Col I⁺ cells represented 0.017% of total cells in the lungs of both LPA₁ KO and WT mice before bleomycin challenge. Seven days following bleomycin challenge, the quantities of CD45⁺ Col I⁺ cells in the lungs increased similarly in both genotypes: to 0.55% of lung cells in WT mice, and to 0.58% of lung cells in LPA₁ KO mice.

FIG. 14 presents LPA₁ receptor expression in endothelial cell lines.

FIG. 14a demonstrates LPA₁ receptor expression in the C166 mouse endothelial cell line. Data are presented as copies of receptor mRNA relative to copies of GAPDH mRNA. Receptor expression was determined by measuring mRNA using quantitative polymerase chain reaction.

FIG. 14b demonstrates LPA₁ receptor expression in primary mouse cardiac endothelial cells. Data are presented as copies of receptor mRNA relative to copies of GAPDH mRNA. Receptor expression was determined by measuring mRNA using quantitative polymerase chain reaction.

FIG. 15 presents lung fibrin turnover induced by bleomycin injury as assessed by BAL D-dimer concentration. n=7 or 8 WT and LPA₁^{-/-} mice at each time point, except n=3 at D0. Data presented are pooled from two independent experiments with similar results, and are expressed as mean BAL D-dimer concentration+SEM. * P=0.0087, LPA₁^{-/-} D5 versus WT D5.

FIG. 16 presents exemplary data showing TGF-β1 levels in WT and LPA₁ KO mice before and after bleomycin challenge. Total TGF-β1 levels in

FIG. 16a shows total TGF-β1 in BAL samples from WT and LPA₁ KO mice before bleomycin challenge and on Day 5, Day 7 and Day 14 after bleomycin challenge.

FIG. 16b shows total TGF-β1 in lung homogenates from WT and LPA₁ KO mice before bleomycin challenge and on Day 5 after bleomycin challenge. TGF-β1 levels were determined by commercially available ELISA (R&D Systems) according to the manufacturer's instructions. Total TGF-β1 levels were determined following activation of latent TGF-β1 to the immunoreactive form detectable by this ELISA by acidification of samples with HCl and then neutralization with NaOH/HEPES. Increases in TGF-β levels in BAL and lung homogenates induced by bleomycin injury in WT mice were reduced in LPA₁ KO mice, although the differences between genotypes did not reach statistical significance.

FIG. 17 presents one embodiment of a human LPA₁ nucleotide sequence (SEQ ID NO:1) (Accession No. NM_{—}057159).

FIG. 18 presents one embodiment of a human LPA₁ amino acid sequence (SEQ ID NO:2) (Accession No. NM_{—}057159).

FIG. 19 presents one embodiment of a human LPA₁ nucleotide sequence (SEQ ID NO:3) (Accession No. NM_{—}001401).

FIG. 20 presents one embodiment of a human LPA₁ amino acid sequence (SEQ ID NO:4) (Accession No. NM_{—}001401).

FIG. 21 presents one embodiment of: FIG. 21A—A human LPA₁ nucleotide sequence (SEQ ID NO:5); and FIG. 21B—A human LPA₁ amino acid sequence (SEQ ID NO: 6) (Accession No. NM_{—}012152).

FIG. 22 presents one embodiment of a mouse LPA₁ nucleotide sequence (SEQ ID NO:7) (Accession No. NM_{—}010336).

FIG. 23 presents one embodiment of a mouse LPA₁ amino acid sequence (SEQ ID NO:8) (Accession No. NM_{—}010336).

### DETAILED DESCRIPTION

The present invention is related to the treatment of fibrotic diseases. For example, a fibrotic disease may include, but is not limited to, a pulmonary disease characterized by the generation of lysophosphatidic acid (LPA). The present invention contemplates methods and compositions related to the effective treatment of fibrotic lung diseases by administering inhibitory compounds directed to an LPA receptor. For example, one such receptor comprises LPA₁.

In one embodiment, the present invention contemplates a method for identifying chemoattractant(s) that direct fibroblast migration during pulmonary fibrosis. In one embodiment, the fibroblast migration occurs within lung airspaces. Although it is not necessary to understand the mechanism of an invention it is believed that fibroblast chemotactic activity of BAL samples collected from bleomycin-injured mice co-purifies with albumin. It is further believed that bleomycin-induced BAL sample chemoattractant activity may be attributable to albumin-bound LPA. The data presented herein establishes that lysophosphatidic acid (LPA) is a chemotactic factor during primary lung fibroblasts, and that LPA is generated in lung airspaces following bleomycin injury. The data further shows that the LPA₁ receptor plays a role in mediating LPA activity that may be responsible for the development of pulmonary fibrosis. For example, when LPA₁-deficient mice are challenged with bleomycin, these mice have a reduced incidence of pulmonary fibrosis. In addition to mitigating the excessive accumulation of fibroblasts in an injured lung, the absence of LPA₁ receptors markedly reduces vascular leak usually produced by lung injury. These data indicate that the LPA₁ receptor may mediate LPA effects relevant to aberrant wound-healing responses that may be responsible for the development of pulmonary fibrosis.

### I. Lung Injury Physiology

A. Fibrosis

As seen in cutaneous injuries, fibroblasts migrate into the fibrin-rich exudates that develop in lung alveoli (i.e., for example, airspaces) following lung injury in both acute respiratory distress syndrome (ARDS) and idiopathic pulmonary fibrosis (IPF). Kuhn et al., “An immunohistochemical study of architectural remodeling and connective tissue synthesis in pulmonary fibrosis” Am Rev Respir Dis 140:1693-1703 (1989). Concomitantly, these diseases result in increased fibroblast chemoattractant activity within lung airspaces. Snyder et al., “Acute lung injury. Pathogenesis of intraalveolar fibrosis” Journal of Clinical Investigation 88:663-73 (1991); and Behr et al., “Fibroblast chemotactic response elicited by native bronchoalveolar lavage fluid from patients with fibrosing alveolitis” Thorax 48:736-742. (1993).

Evidence suggesting that inhibition of fibroblast migration can attenuate the development of pulmonary fibrosis has been recently reported. Tager et al., “Inhibition of pulmonary fibrosis by the chemokine IP-10/CXCL10” Am J Respir Cell Mol Biol 31:395-404 (2004); Phillips et al., “Circulating fibrocytes traffic to the lungs in response to CXCL12 and mediate fibrosis” J Clin Invest 114:438-446 (2004); and Moore et al., “The Role of CCL12 in the Recruitment of Fibrocytes and Lung Fibrosis” Am. J. Respir. Cell Mol. Biol. 35:175-181 (2006).

In one embodiment, the present invention contemplates a method for treating lung fibrosis developing in response to such injury in diseases including, but not limited to, ARDS and IPF.

Fibroblast chemoattractant activity is believed to be generated in the airspaces (i.e., for example, alveoli) in IPF, and positively correlates with disease severity. Further, fibroblast chemoattractant activity has previously been demonstrated to be generated in the airspaces of IPF patients, and the extent of this activity has been found to inversely correlate with patients' total lung capacity and vital capacity. Behr et al. “Fibroblast chemotactic response elicited by native bronchoalveolar lavage fluid from patients with fibrosing alveolitis” Thorax 48:736-742 (1993). A pathogenic role for fibroblast migration in IPF has been further supported by the recent description of an accelerated variant of IPF. Genes related to cell migration were upregulated in the lungs of these “rapid” progressors, and BAL samples from these patients induced significantly greater fibroblast migration than BAL samples from “slow” progressors. In a recent evaluation of the clinical and molecular features of “rapid” and “slow” progressors with IPF, evidence of increased fibroblast migration was associated with an accelerated clinical course and higher mortality. Genes related to cell migration were upregulated in the lungs of “rapid” progressors, defined by their presentation to medical attention <6 months after the onset of symptoms, and BAL samples from these patients induced significantly greater fibroblast migration than BAL from “slow” progressors“, defined by their presentation to medical attention ≧24 months after symptom onset. Selman et al., “Accelerated variant of idiopathic pulmonary fibrosis: clinical behavior and gene expression pattern” PLoS ONE 2, e482 (2007). In one embodiment, the present invention contemplates that LPA is a mediator of fibroblast migration generated in response to an injured lung.

Recently reported evidence suggests that inhibition of chemokine-mediated fibroblast migration can inhibit the development of pulmonary fibrosis. Tager et al., “Inhibition of pulmonary fibrosis by the chemokine IP-10/CXCL10” Am J Respir Cell Mol Biol 31:395-404 (2004); Phillips et al., “Circulating fibrocytes traffic to the lungs in response to CXCL12 and mediate fibrosis” J Clin Invest 114:438-446 (2004); and Moore et al., “The Role of CCL12 in the Recruitment of Fibrocytes and Lung Fibrosis” Am J Respir Cell Mol Biol 35:175-181 (2006).

Fibroblast migration into the fibrin provisional wound matrix is believed to play a role in wound healing responses to injury in multiple tissues. Martin, P. “Wound healing—aiming for perfect skin regeneration” Science 276:75-81 (1997). Some research has included observation in the lung, in which fibroblasts migrate into the fibrin-rich exudates that develop in the alveoli following lung injury. Basset et al., “Intraluminal fibrosis in interstitial lung disorders” American Journal of Pathology 122:443-61 (1986). The data presented herein demonstrate that LPA is one chemoattractant inducing fibroblast migration in the injured lung. LPA recently has been demonstrated to direct the migration of cancer cells, playing a role in cancer pathophysiology by specifically inducing the invasion of cancer cells across tissue barriers and promoting metastasis. Mills et al., “The emerging role of lysophosphatidic acid in cancer” Nat Rev Cancer 3:582-91 (2003). Although it is not necessary to understand the mechanism of an invention it is believed that that LPA may play an analogous role by directing the invasion of fibroblasts across the alveolar basement membrane into the provisional extracellular matrix that is present in the airspaces following lung injury. Several adhesion molecules have been implicated in this process, including CD44 and β1 integrins. Svee et al., “Acute lung injury fibroblast migration and invasion of a fibrin matrix is mediated by CD44” J Clin Invest 98:1713-1727. (1996); and White et al., “Integrin alpha₄beta₁ regulates migration across basement membranes by lung fibroblasts: a role for phosphatase and tensin homologue deleted on chromosome 10” Am J Respir Crit Care Med 168:436-442 (2003), respectively). No chemoattractant(s), however, have yet been identified to direct basement membrane invasion.

In one embodiment, the present invention contemplates a method for inhibiting lung fibroblast recruitment by administering an LPA₁ receptor inhibitor. In one embodiment, the inhibitor blocks LPA signaling, thereby reducing fibroblast invasion across basement membranes and into fibrin matrix. In one embodiment, the LPA₁ receptor inhibitor partially inhibits total fibroblast recruitment. Although it is not necessary to understand the mechanism of an invention it is believed that other fibroblast chemoattractants in addition to LPA are generated following injury, including, but not limited to, chemokines such as CXCL12 and CCL12. Phillips et al., “Circulating fibrocytes traffic to the lungs in response to CXCL12 and mediate fibrosis” J Clin Invest 114:438-446 (2004); and Moore et al., “The Role of CCL12 in the Recruitment of Fibrocytes and Lung Fibrosis” Am. J. Respir. Cell Mol. Biol. 35:175-181 (2006), respectively). These chemokines are believed to direct the trafficking of extrapulmonary mesenchymal precursors into the lung following injury, and LPA could act cooperatively with these chemokines by directing the invasion of these cells, or the fibroblasts they produce, into lung airspaces.

The deposition of fibrin has been suggested to be caused by persistant vascular leak (i.e., for example, increased vascular permeability) during the development of lung injury fibrosis. Chambers et al., “Coagulation cascade proteases and tissue fibrosis” Biochem Soc Trans 30: 194-200 (2002). This increased vascular permeability may cause fibrinogen to extravasate along with other plasma proteins into lung airspaces, thereby activating a clotting cascade. Whereas fibrin is not usually present in normal lung tissue, fibrin deposition has been observed following bleomycin-induced injury. Olman et al., “Changes in procoagulant and fibrinolytic gene expression during bleomycin-induced lung injury in the mouse” J Clin Invest 96:1621-1630 (1995). Lung fibrin deposition is also characteristic of: i) ARDS, in which intraalveolar fibrin lines denuded alveolar epithelium (Bachofen et al., “Structural alterations of lung parenchyma in the adult respiratory distress syndrome” Clin Chest Med 3:35-56 (1982); and ii) IPF, in which fibrin is deposited in areas of active fibrosis. Imokawa et al., “Tissue factor expression and fibrin deposition in the lungs of patients with idiopathic pulmonary fibrosis and systemic sclerosis” Am J Respir Crit Care Med 156:631-636 (1997). The contribution of excessive or excessively persistent fibrin to the development of fibrosis has been demonstrated by multiple studies that have examined the effects of increasing or decreasing fibrin accumulation on bleomycin-induced pulmonary fibrosis. Eitzman et al., “Bleomycin-induced pulmonary fibrosis in transgenic mice that either lack or overexpress the murine plasminogen activator inhibitor-1 gene” J Clin Invest 97:232-237 (1996); and Swaisgood et al., “The development of bleomycin-induced pulmonary fibrosis in mice deficient for components of the fibrinolytic system” Am J Pathol 157:177-187 (2000).

In one embodiment, the present invention contemplates a method of inhibiting pulmonary fibrosis by reducing fibrin deposition in injured lung airspaces. In one embodiment, the fibrin deposition is determined by measuring D-dimer levels. In one embodiment, the inhibiting comprises administering a LPA₁ receptor inhibitor. In one embodiment, the D-dimers are generated from fibrin that were crosslinked during a coagulation process. In one embodiment, the method further comprises inhibiting vascular leak thereby further reducing fibrin deposition.

B. Vascular Permeability

Tissue injury is usually associated with increased vascular permeability. Martin, P. “Wound healing—aiming for perfect skin regeneration” Science 276:75-81 (1997). It has been reported that release of bioactive mediators may be responsible for increased vascular permeability (i.e., for example, vascular leak) observed during the early phases of tissue repair. Dvorak, H. F., “Tumors: wounds that do not heal. Similarities between tumor stroma generation and wound healing” N Engl J Med 315:1650-1659 (1986). For example, increased transport of fluid and macromolecules across the endothelium may occur under pathologic conditions (i.e., for example, lung injury). Some believe that this fluid transport occurs through paracellular gaps formed by the disruption of endothelial intercellular junctions. Dudek et al., “Cytoskeletal regulation of pulmonary vascular permeability” J Appl Physiol 91:1487-1500 (2001). In vitro studies have suggested that LPA may play a role in endothelial barrier dysfunction by inducing actin stress fiber formation thereby resulting in the development of paracellular gaps. van Nieuw Amerongen et al., “Role of RhoA and Rho kinase in lysophosphatidic acid-induced endothelial barrier dysfunction” Arterioscler Thromb Vasc Biol 20:E127-E133 (2000).

Extravascular coagulation may be one consequence of persistent vascular leak induced by lung injury that may contribute to the development of fibrosis. Idell S., “Coagulation, fibrinolysis, and fibrin deposition in acute lung injury” Critical Care Medicine 31:S213-220 (2003); and Chambers et al., “Coagulation cascade proteases and tissue fibrosis” Biochem Soc Trans 30:194-200 (2002). Increased vascular permeability may cause coagulation cascade proteins to extravasate into the lung airspaces, where they could be activated by tissue procoagulants. A resultant deposition of fibrin is thought to provide a provisional matrix through which fibroblasts migrate during tissue repair. Fibrin deposition in the lung airspaces may also promote epithelial-to-mesenchymal transition, further contributing to fibroblast accumulation and eventual fibrosis development. Loskutoff et al., “PAI-1, fibrosis, and the elusive provisional fibrin matrix” J Clin Invest 106:3 (2000).

Coagulation cascade proteins (i.e., for example, thrombin) in addition to generating fibrin, also activate protease activated receptors (PARs). PARs may also promote fibrosis independently of fibrin generation through the induction of mediators such as PDGF. Chambers et al., “Coagulation cascade proteases and tissue fibrosis” Biochem Soc Trans 30:194-200 (2002). Therefore, although the mechanisms are not yet completely understood, excessive extravascular coagulation may promote lung fibrosis following injury.

In one embodiment, the present invention contemplates a method for inhibiting vascular leak by the administration of an LPA, receptor inhibitor. In one embodiment, the receptor inhibitor reduces LPA signaling by endothelial cells. In one embodiment, the vascular leak occurs in vivo following a lung injury. Although it is not necessary to understand the mechanism of an invention it is believed that LPA may act in opposition to other lysophospholipids including, but not limited to, sphingosine 1-phosphate (S1P). It is believed that S1P signals through S1P¹⁻⁵ GPCRs some of which may share homology with LPA receptors. Ishii et al., “Lysophospholipid receptors: signaling and biology” Annu Rev Biochem 73:321-54 (2004). In contrast to LPA, SIP appears to strengthen endothelial intercellular junctions and enhances endothelial barrier integrity, thereby acting to reduce vascular leakage. Garcia et al., “Sphingosine 1-phosphate promotes endothelial cell barrier integrity by Edg-dependent cytoskeletal rearrangement” J Clin Invest 108:689-701 (2001).

C. Leukocyte Recruitment And/Or Migration

Leukocyte recruitment and/or migration (i.e., for example, chemotaxis) was not affected when LPA₁^{-/-} mice were challenged with bleomyin. This response is in contrast to reduced fibroblast recruitment and reduced vascular leak observed in LPA₁^{-/-} mice. Although it is not necessary to understand the mechanism of an invention it is believed that leukocyte recruitment may occur independently of an LPA₁ receptor. For example, the generation of inflammatory leukocyte responses in LPA₁^{-/-} mice indicate that inflammatory and fibrotic responses to lung injury are uncoupled in the absence of LPA₁ expression. This proposed dissociation between leukocyte-induced inflammation and fibroblast-induced fibrotic responses is consistent with other observations using mice deficient for the β6 integrin. Munger et al., “The integrin alphaᵥbeta₆ binds and activates latent TGFβ₁: a mechanism for regulating pulmonary inflammation and fibrosis” Cell 96:319-328 (1999). A dissociation of fibrosis from inflammation in lung injury, suggest that inflammation need not play a role in the development of pulmonary fibrosis.

II. Lysophosphatidic Acid

Lysophosphatidic acid (LPA) has potent fibroblast chemoattractant properties. Kundra et al., “The chemotactic response to PDGF-BB: evidence of a role for Ras” J Cell Biol 130:725-731 (1995). LPA, however, also induces endothelial cell barrier dysfunction and vascular leak. van Nieuw Amerongen et al., “Role of RhoA and Rho kinase in lysophosphatidic acid-induced endothelial barrier dysfunction” Arterioscler Thromb Vase Biol 20:E127-E133 (2000); and Neidlinger et al., “Hydrolysis of phosphatidylserine-exposing red blood cells by secretory phospholipase A2 generates lysophosphatidic acid and results in vascular dysfunction” J Biol Chem 281:775-781 (2006). Further, vascular permeability is increased throughout the early phases of tissue repair following injury. Dvorak, H. F., “Tumors: wounds that do not heal. Similarities between tumor stroma generation and wound healing” N Engl J Med 315:1650-1659 (1986). For example, in the injured lung, endothelial barrier integrity is disrupted by: i) paracellular gap formation; ii) persistent vascular leak leading to extravasation of plasma proteins; and iii) coagulation and fibrin deposition in the airspaces. Dudek et al., “Cytoskeletal regulation of pulmonary vascular permeability” J Appl Physiol 91:1487-1500 (2001).

Fibroblast migration chemoattractant properties were studied by biophysically purifying a fibroblast chemoattractant activity present in lung airspaces following bleomycin-induced injury (i.e., for example, the bleomycin mouse model of pulmonary fibrosis). The data suggest that fibroblast migration induced by lung injury is mediated by LPA, acting through one of its specific G protein-coupled receptors (GPCRs), LPA₁.

To evaluate whether LPA utilizes the LPA₁ pathway in the development of pulmonary fibrosis, lung injury was induced using a bleomycin model in LPA₁-deficient mice (i.e., LPA₁^{-/-} mice). The data shows that, following bleomycin administration, LPA₁^{-/-} mice were protected from fibrosis, exhibited greatly diminished vascular leak, and showed reduced fibroblast recruitment. In one embodiment, the present invention contemplates that an LPA₁ receptor comprises an inhibitory drug target capable of preventing lung injury and the subsequent development of pulmonary fibrosis.

The increased levels of LPA that are present in the airspaces following bleomycin injury may be derived from several different sources, including platelets and surfactant. Platelet-derived LPA has recently been shown to support the progression of osteolytic bone metastases in breast and ovarian cancer. Boucharaba et al., “Platelet-derived lysophosphatidic acid supports the progression of osteolytic bone metastases in breast cancer” J. Clin. Invest. 114:1714-1725 (2004). Platelet activation has been reported to occur in lung airspaces of patients with IPF and ARDS. Idell et al., “Platelet-specific alpha-granule proteins and thrombospondin in bronchoalveolar lavage in the adult respiratory distress syndrome” Chest 96:1125-1132 (1989). Recently, platelet activation has been suggested to play a role in the development of lung injury vascular leakage. Zarbock et al., “Complete reversal of acid-induced acute lung injury by blocking of platelet-neutrophil aggregation” J Clin Invest 116:3211-3219 (2006).

Alternatively, hydrolysis of pulmonary surfactant phospholipids may produce LPA in ARDS and IPF patients. Gregory et al., “Surfactant chemical composition and biophysical activity in acute respiratory distress syndrome” J Clin Invest 88:1976-1981 (1991); and Honda et al., “Changes in phospholipids in bronchoalveolar lavage fluid of patients with interstitial lung diseases” Lung 166:293-301 (1988), respectively. Phospholipid breakdown is thought to impair a surfactant's ability to decrease surface tension, thereby promoting lung collapse following injury. Hite et al., “Hydrolysis of surfactant-associated phosphatidylcholine by mammalian secretory phospholipases A2” Am J Physiol 275:L740-L747 (1998). Although it is not necessary to understand the mechanism of an invention it is believed that hydrolysis of surfactant phospholipids may also contribute to lung injury through the generation of LPA, which may then direct both vascular leak and fibroblast recruitment.

### III. Characterization Of Lung Fibroblast Chemoattractant(s)

A. Fibroblast Chemotactic Activity And G Protein-Coupled Receptors (GPCRs).

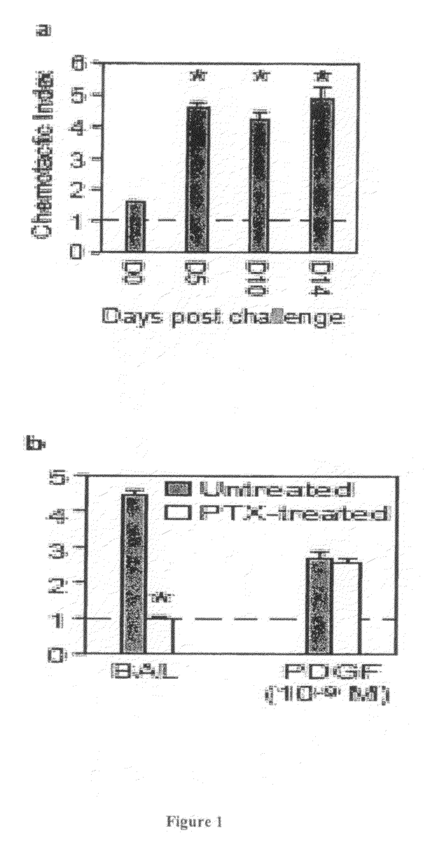

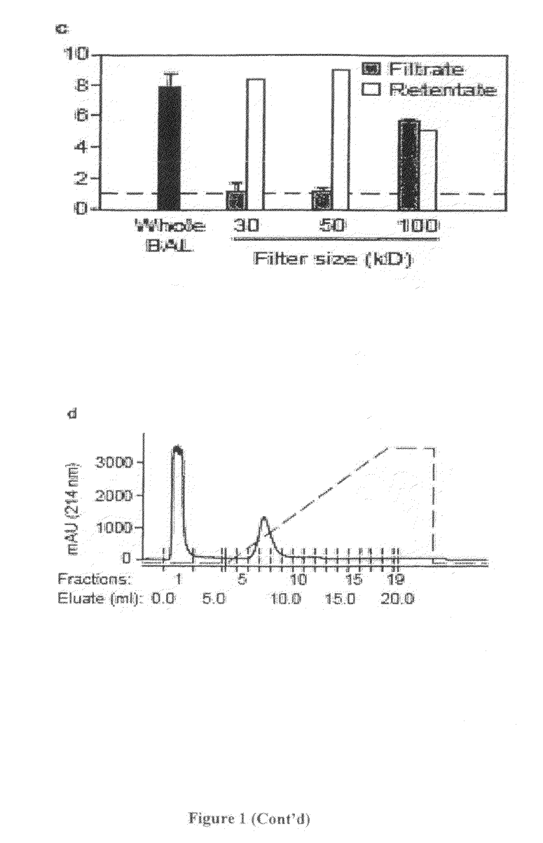

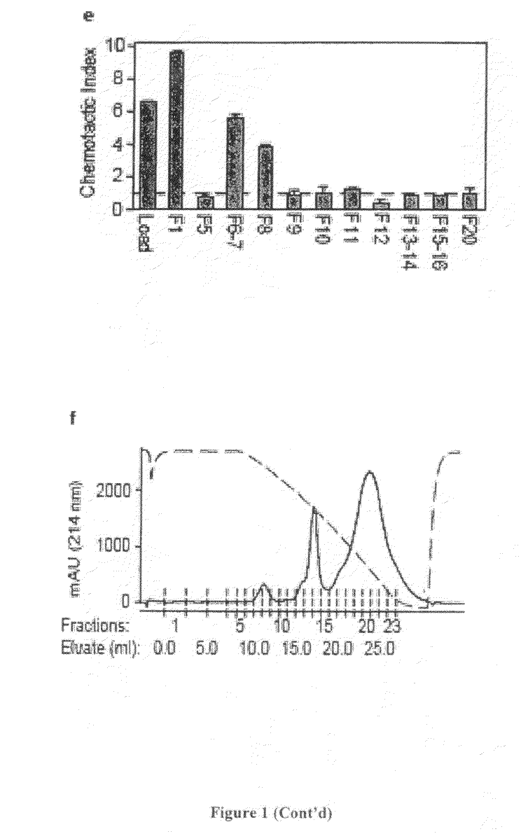

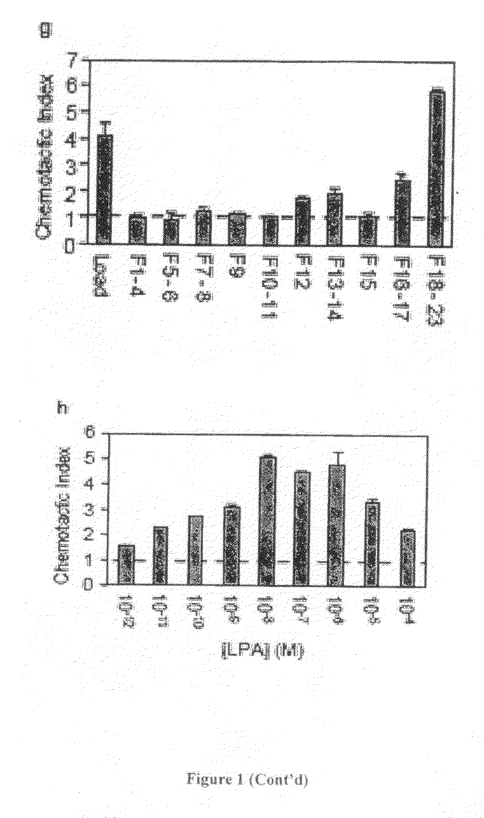

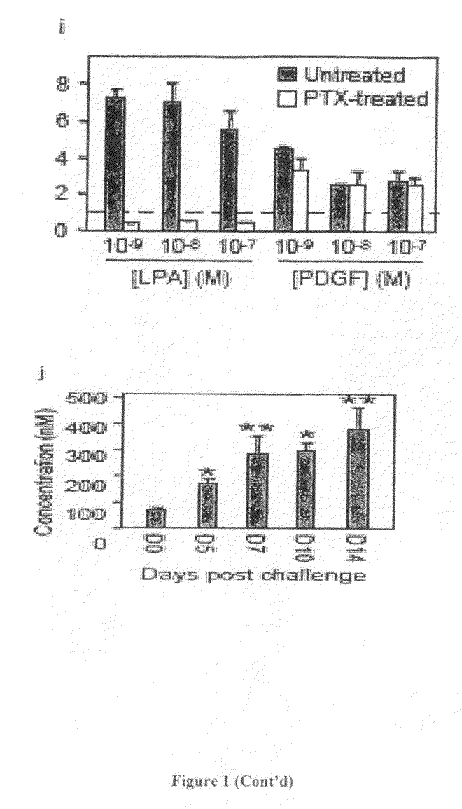

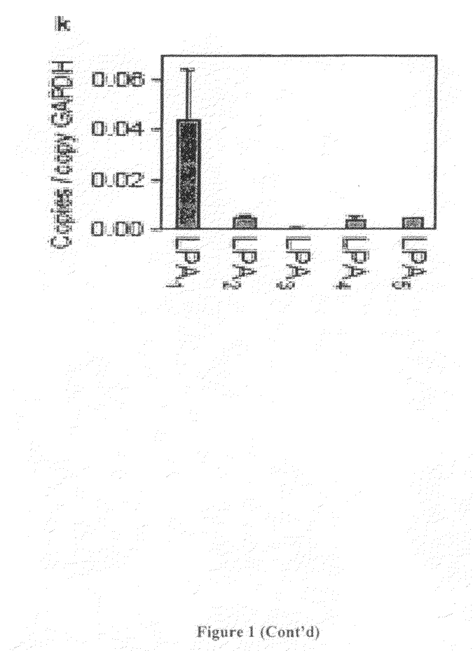

ARDS and IPF is characterized by the appearance of fibroblast chemoattractant activity. Snyder et al., “Acute lung injury. Pathogenesis of intraalveolar fibrosis” Journal of Clinical Investigation 88:663-73 (1991); and Behr et al., “Fibroblast chemotactic response elicited by native bronchoalveolar lavage fluid from patients with fibrosing alveolitis” Thorax 48:736-742.(1993), respectively. Analogously, fibroblast chemoattractant activity appears in the bleomycin model of injury-induced lung fibrosis. The data presented herein was collected following bronchoalveolar lavage (BAL) fluid recovered from bleomycin-exposed mice. The BAL collected from bleomycin-challenged mice attracted primary mouse lung fibroblasts, unlike BAL fluid collected from unchallenged mice. See, FIG. 1a.

In ARDS patients, platelet-derived growth factor (PDGF) or PDGF-related peptides has been reported as being partially responsible for fibroblast chemoattractant activity. Snyder et al., 1991. However, the data presented herein demonstrates that bleomycin-induced fibroblast chemotactic activity is completely inhibited by pertussis toxin (PTX) pretreatment. See, FIG. 1b. Although it is not necessary to understand the mechanism of an invention, it is believed that these data indicate that one relevant fibroblast receptor signal may be mediated by a Gα₁ class of G proteins. Such GPCR proteins have been reported to respond to chemoattractants. Luster A. D., “Chemokines—chemotactic cytokines that mediate inflammation” N Engl J Med 338:436-445 (1998). In contrast, PDGF signals through receptor tyrosine kinases, and fibroblast chemotaxis induced by PDGF was not inhibited by PTX pretreatment. See, FIG. 1b. A wide array of chemoattractants are believed to signal through PTX-sensitive GPCRs, including chemokines, which are induced by lung injury. Strieter et al., “Chemokines in Lung Injury: Thomas A. Neff Lecture” Chest 116:103S-110S (1999).

B. Biophysical Characterization Of Fibroblast Chemoattractants

Fibroblast chemoattractant(s) may be characterized by determining molecular size, heparin binding affinity, and hydrophobicity. The data presented below reveal that the BAL chemoattractant(s) induced by bleomycin administration are not chemokines.

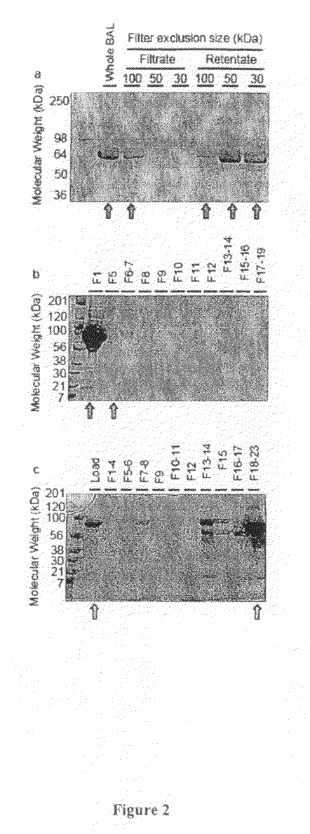

Molecular size was determined by a comparing results using 30, 50, and 100 kDa molecular exclusion filters. When BAL was centrifuged over molecular exclusion filters having sizes of 30 and 50 kDa the retentates had chemotactic activity equivalent to unfractionated BAL, whereas the filtrates had no chemotactic activity. See, FIG. 1c. In contrast, chemotactic activity was present in both the retentate and the filtrate produced by centrifugation of BAL over a 100 kDa filter. These data indicate that the molecule(s) responsible for the chemoattractant activity of BAL fluid have molecular weights between 50 and 100 kDa. SDS-PAGE confirmed that the proteins between 50 and 100 kDa present in the unfractionated BAL fluid were present in the 30 and 50 kDa retentates. However, the 50 and 100 kDa proteins were present in both the retentate and filtrate produced by the 100 kDa filter. See, FIG. 2a. These results suggest that the BAL fibroblast chemoattractants are not chemokines, which are small proteins between 8 to 10 kDa. Luster A. D., “Chemokines--chemotactic cytokines that mediate inflammation” N Engl J Med 338:436-445 (1998).

Heparin binding affinity chromatography of BAL fibroblast chemoattractant(s) showed that the most abundant proteins in BAL either did not bind, or eluted from the heparin affinity column at low NaCl concentrations. See, FIG. 1d. Consequently, these data suggest that BAL fibroblast chemoattractant(s) may have a low heparin binding affinities. For example, proteins present in: i) the flow-through (fraction 1); or ii) elution fractions 6, 7 and 8 demonstrated fibroblast chemotactic activity. See, FIG. 1e. In contrast, the proteins eluting at higher NaCl concentrations did not induce fibroblast chemotactic activity. The SDS-PAGE electrophoresis gel banding pattern indicating the heparin affinity fractions is shown. See, FIG. 2b. These data support the above indication that BAL fibroblast chemoattractant(s) are not chemokines, because chemokines typically have high heparin binding affinities. Luster A. D., “Chemokines—chemotactic cytokines that mediate inflammation” N Engl J Med 338:436-445 (1998).

Hydrophobicity interaction chromatography of BAL fibroblast chemoattractant(s) showed that the most abundant BAL proteins eluted from the hydrophobic interaction column at low (NH₄)₂SO₄ concentrations. See, FIG. 1f. Consequently, these data suggest that BAL fibroblast chemoattractant(s) may have a high surface hydrophobicity. Proteins that eluted in fractions 18-23 demonstrated fibroblast chemotactic activity, whereas proteins with lower hydrophobicity did not induce fibroblast chemotaxis. See, FIG. 1g. The SDS-PAGE electrophoresis gel banding pattern indicating the hydrophobic interaction fractions is shown. See, FIG. 2c. The data support the above indication that BAL fibroblast chemoattractant(s) are not chemokines, because chemokines are typically highly charged basic proteins.

Consequently, the data from molecular size exclusion, heparin binding chromatography, and hydrophobicity interaction chromatography support the conclusion that bleomycin-induced BAL chemoattractant(s) are not chemokines. Further, these biophysical separations indicate that the BAL sample comprises fibroblast chemoattractant(s) with low heparin affinity, high hydrophobicity, and molecular weights ranging between 50 and 100 kD. It is also possible that BAL samples contain fibroblast chemoattractants other than chemokines (typically being highly charged basic proteins between 8 to 10 kD in size and have high heparin affinity).

C. Albumin-Bound LPA May Represent Chemoattractant Activity

All BAL sample separation fractions discussed above all with chemotactic activity contained an abundant protein the size of mouse albumin (i.e., for example, approximately 69 kDa) as visualized by SDS-PAGE electrophoresis. See, FIG. 2. Albumin is believed to transport lipids via hydrophobic binding. Curry et al., “Fatty acid binding to human serum albumin: new insights from crystallographic studies” Biochim Biophys Acta 1441:131-140 (1999). Consequently, it was reasonable to hypothesize that a lipid-albumin complex may be responsible for BAL fibroblast chemoattractant activity. Albumin co-purifying with serum activity that stimulates fibroblast actin stress fiber and focal adhesion formation was attributed to albumin-bound LPA. Ridley et al., “The small GTP-binding protein rho regulates the assembly of focal adhesions and actin stress fibers in response to growth factors” Cell 70:389-99 (1992). Since cytoskeletal actin rearrangement may be involved in cell migration, it was suspected that LPA may mediate bleomycin-induced BAL fluid fibroblast migration.

1. LPA Chemotaxis Is Mediated By a GPCR

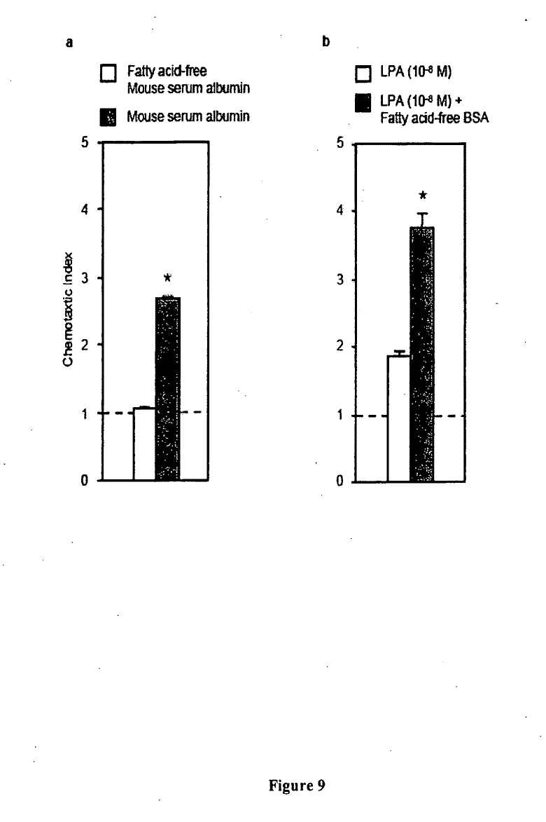

The data presented herein demonstrates that LPA bound to albumin was chemotactic for primary mouse lung fibroblasts. See, FIG. 1h. LPA-induced fibroblast chemotaxis was reduced by PTX pre-treatment, confirming that the relevant LPA receptor(s) are GPCRs. See, FIG. 1i. Further, methanol-extracted fatty acid-free albumin did not induce fibroblast chemotaxis. See, FIG. 9a. But methanol extracted fatty acid-free albumin significantly potentiated LPA chemotactic activity. FIG. 9b. The processes involved in the formation of serum from blood, particularly platelet activation, generate LPA. Serum albumin preparations that have not had bound lipids extracted therefore contain LPA, and would be expected to induce fibroblast chemotaxis, as observed.

These data confirm that LPA receptor(s) responsible for mediating the activity of bleomycin-induced fibroblast chemoattractant(s) are GPCRs.

2. Generation Of LPA In Bleomycin-Induced BAL Fluid

The data presented herein show that LPA is generated in the airspaces following bleomycin-induced lung injury. LPA in BAL fluid was determined by electrospray ionization mass spectrometry (ESI-MS). LPA in BAL fluid from unchallenged mice (Day 0) was compared to BAL fluid after a bleomycin challenge (Days 5, 7, 10, and 14). The data show that the LPA concentration in BAL fluid was significantly elevated at all time points following bleomycin-induced lung injury. See, FIG. 2j.

In conclusion, the above data show that LPA is not only present in post-bleomycin challenge BAL fluid but LPA is a potent fibroblast chemoattractant. In one embodiment, the present invention contemplates a method of treating lung injury comprising inhibiting LPA-induced fibroblast chemoattractant activity.

3. Characterization Of The LPA Chemoattractant Receptor Subtype

To identify the relevant LPA receptor(s), LPA receptor expression in primary mouse lung fibroblasts was subjected to Quantitative Polymerase Chain Reaction (QPCR). LPA is believed to have at least five different receptor subtypes. For example, these subtypes may include, but are not limited to, a series of GPCRs designated LPA₁₋₅. Ishii et al., “Lysophospholipid receptors: signaling and biology” Annu Rev Biochem 73:321-54 (2004); Noguchi et al., “Identification of p2y9/GPR23 as a novel G protein coupled receptor for lysophosphatidic acid, structurally distant from the Edg family”. J Biol Chem 278:25600-25606 (2003); and Lee et al., “GPR92 as a new G(12/13)-and G(q)-coupled lysophosphatidic acid receptor that increases cAMP, LPA₅” J Biol Chem 281:23589-23597 (2006).

The data presented herein show that lung fibroblasts express a high proportion of the LPA₁ receptor subtype. See, FIG. 1k. The LPA₁ receptor subtype, has been reported to mediate LPA-induced chemotaxis of mouse embryonic fibroblasts. Hama et al., “Lysophosphatidic acid and autotoxin stimulate cell motility of neoplastic and non-neoplastic cells through LPA₁” J Biol Chem 279:17634-17639 (2004). In one embodiment, the present invention contemplates a method of inducing BAL-induced fibroblast chemotaxis by LPA signaling through a LPA₁ receptor subtype.

D. LPA₁ Receptors Mediate Bleomycin-Induced Pulmonary Fibrosis

The role of LPA₁ in mediating LPA-directed fibroblast recruitment in vivo was further studied by using mice genetically deficient for LPA₁ (i.e., LPA₁^{-/-} mice). Contos et al., “Requirement for the IpA1 lysophosphatidic acid receptor gene in normal suckling behavior” Proc Natl Acad Sci USA 97:13384-13389 (2000).

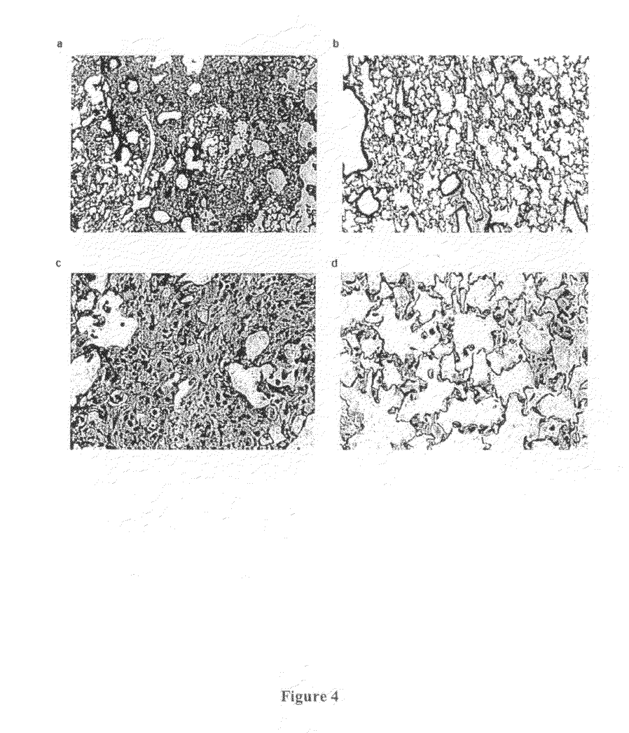

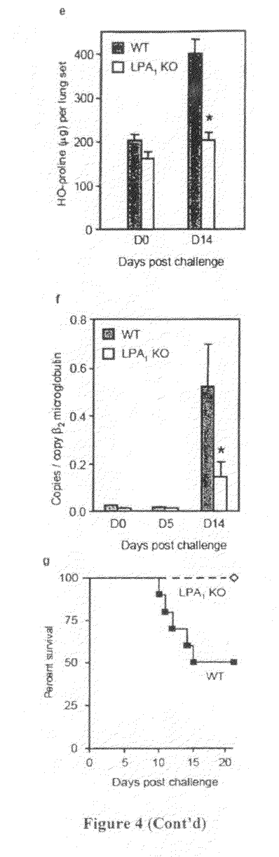

Histologic analysis of the lungs of wild type and LPA₁-deficient mice (i.e., LPA₁^{-/-}) 14 days following a bleomycin challenge demonstrated that LPA₁-deficient mice were markedly from bleomycin-induced fibrosis. Examination of lung tissue at 14 days post-bleomycin challenge typically demonstrate changes consistent with peribronchiolar and parenchymal fibrosis. The extent of these changes present in wild type mice was substantially decreased in LPA₁-deficient mice. See, FIG. 4a as compared to FIG. 4b. The amount of lung collagen visualized by Masson's trichrome staining of wild type mice 14 days following bleomycin was also substantially decreased in LPA₁-deficient mice. See, FIG. 4c as compared to FIG. 4d.

A biochemical assessment of the extent of fibrosis produced 14 days postchallenge in wild type and LPA₁-deficient mice confirmed the protection of LPA₁-deficient mice. For example, compared to unchallenged mice of the same genotype, the amount of hydroxyproline present in the lungs 14 days following bleomycin challenge increased by 96% in wild type mice, but increased by only 25% in LPA₁-deficient mice. See, FIG. 4e. A statistical interaction (ANOVA) between the effects of genotype and bleomycin treatment on lung hydroxyproline was highly significant at P=0.0073.

Consistent with these hydroxyproline results indicative of reduced collagen protein, bleomycin-challenged LPA₁^{-/-} mice also demonstrated reduced lung collagen gene expression. mRNA levels of the α1 chain of procollagen type I increased in the lungs of both genotypes 14 days after bleomycin challenge, but this increase was reduced in LPA₁^{-/-} mice. See, FIG. 4f.

Finally, at the highest dose of bleomycin (3 units/kg), the absence of LPA1 expression protected mice from mortality: at 21 days post-challenge, the mortality of wild type mice was 50%, whereas the mortality of LPA₁^{-/-} mice was 0% (P 0.0115). See, FIG. 4g.

E. LPA₁ Receptors Mediate Fibroblast Chemotaxis

In one embodiment, the present invention contemplates a method for inhibiting an LPA₁ receptor under conditions such that fibroblast migration is reduced. In one embodiment, the present invention contemplates a method for inhibiting an LPA₁ receptor under conditions such that fibroblast accumulation is reduced.

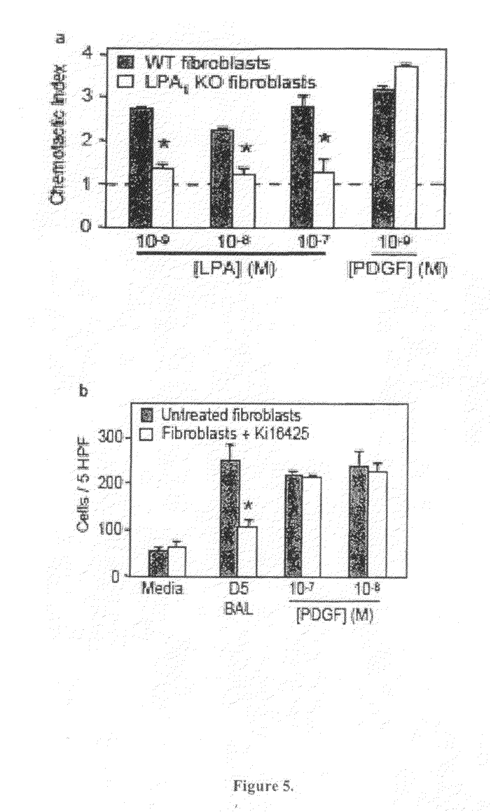

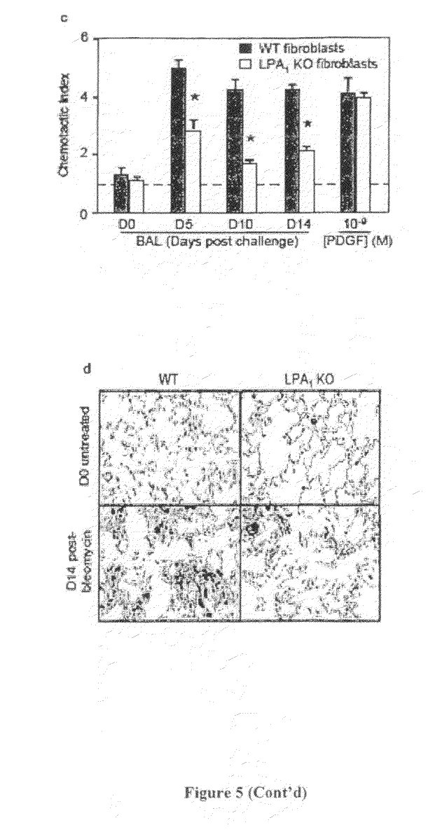

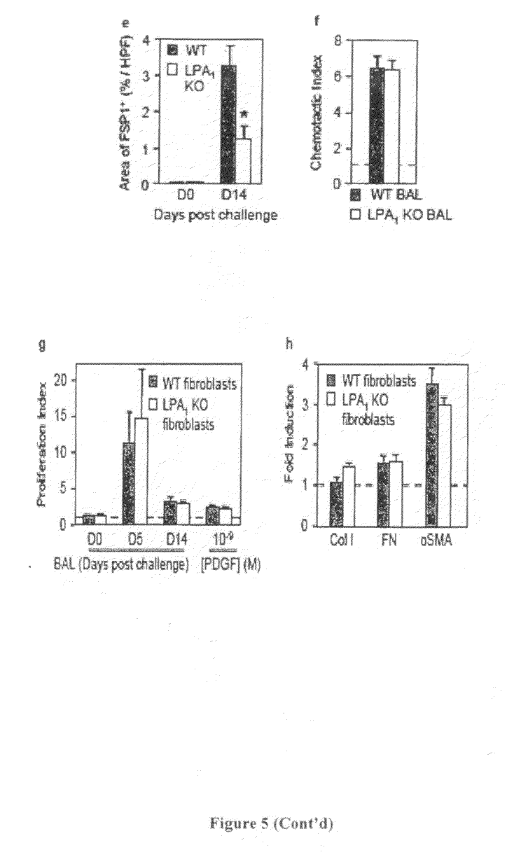

The data presented herein confirms that LPA₁ mediates LPA-induced chemotaxis of lung fibroblasts. For example, in comparison to wild type fibroblasts, fibroblasts isolated from LPA₁^{-/-} mice showed reduced chemotaxis induced by bleomycin-induced BAL samples. In contrast, no differences were observed between wild type and LPA₁^{-/-} fibroblast chemotaxis induced by PDGF. See, FIG. 5a. For example, data collected on Day 10 after the bleomycin challenge, showed the LPA₁^{-/-} fibroblast response to be 25% of the wild type fibroblast response. Similarly, data collected on Day 14 after the bleomycin challenge, showed the LPA₁^{-/-} fibroblast response to be 33% of the wild type fibroblast response. See, FIG. 5a. PDGF-induced chemotaxis was again similar for wild type and LPA₁-deficient fibroblasts. See, FIG. 5a. Other experiments demonstrated that a bleomycin-induced BAL sample chemotactic response in LPA₁-deficient fibroblasts on Day 5 after bleomycin challenge was 45% of wild type response (data not shown). For all time points, the response of LPA₁-deficient fibroblasts to bleomycin-induced BAL sample chemoattractant activity was less than 50% of the response of wild type fibroblasts, indicating that LPA plays a role as a fibroblast chemoattractant generated in the injured lung, in addition to other compounds.

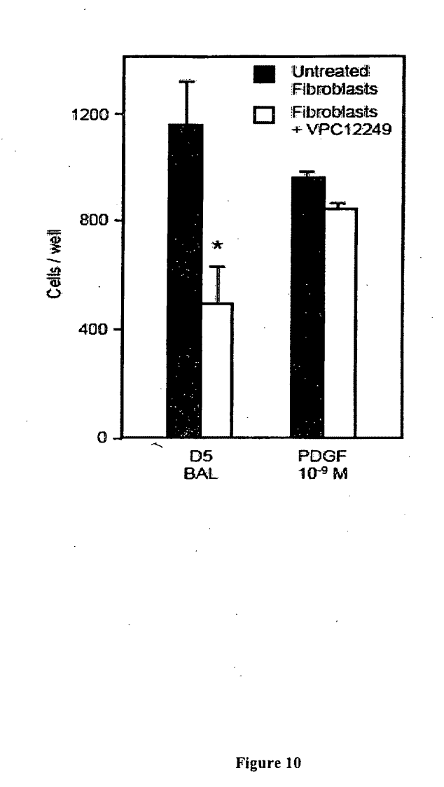

It has been determined that LPA₁ mediates most, but not all, of the total fibroblast chemotactic response following bleomycin-induced lung injury. For example, the LPA receptor antagonist Ki16425 significantly inhibited fibroblast chemotaxis to BAL samples from mice on Day 5 post-bleomycin challenge. See FIG. 5b. PDGF-induced chemotaxis was similar for untreated and Ki16425-treated fibroblasts. See, FIG. 5b. VPC12249, another specific LPA antagonist, also significantly inhibited fibroblast chemotaxis to BAL samples for mice on Day 5 post-bleomycin challenge. See, FIG. 10. PDGF-induced chemotaxis was similar for untreated and VPC12249-treated fibroblasts. See, FIG. 10.

Similarly, chemotaxis induced by BAL samples from bleomycin-challenged mice was reduced with the responding cells were LPA₁^{-/-} fibroblasts. For example, the chemotactic response of LPA1−/− fibroblasts was 45%, 25% and 33% of the response of wild type fibroblasts to BAL samples from mice on Day 5, Day 10 and Day 14 following bleomycin administration, respectively. See, FIG. 5c. Thus, at all time points following bleomycin challenge, the response of LPA₁^{-/-} fibroblasts to the BAL sample chemoattractant activity was less that 50% of the response of wild type fibroblasts. PDGF-induced chemotaxis was again similar for wild-type and LPA₁-deficient fibroblasts. See, FIG. 5c. Given the specificity of LPA₁ for LPA, these data indicate that LPA plays a role as a fibroblast chemoattractant recovered from lung airspaces during the development of bleomycin-induced fibrosis. Ishii et al., “Lysophospholipid receptors: signaling and biology” Annu Rev Biochem 73:321-354 (2004).

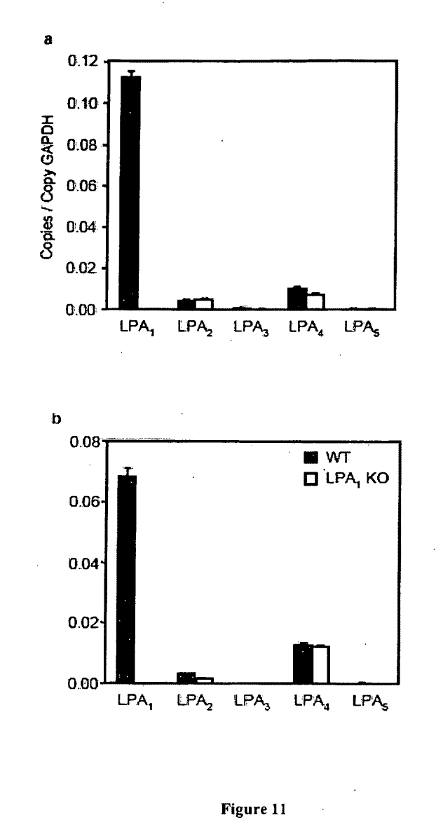

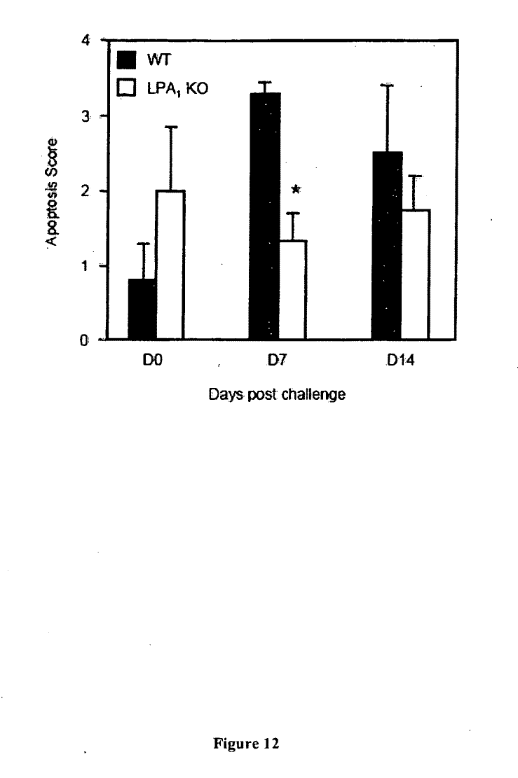

The above data suggests that LPA may be a fibroblast chemoattractant generated in an injured lung. Consequently, the accumulation of fibroblasts might be expected to be attenuated in the lungs of LPA₁-deficient mice following bleomycin-induced injury. For example, LPA₁ remains highly expressed by lung fibroblasts following bleomycin injury, and LPA₁ deficiency does not cause compensatory changes in the expression levels of other LPA receptors in lung fibroblasts. LPA₁ was the most highly expressed LPA receptor in wild type fibroblasts both before bleomycin challenge and on Day 14 after bleomycin challenge. LPA₁ was not expressed by fibroblasts isolated from LPA₁^{-/-} mice at either time point. Expression of LPA₂, LPA₃, LPA₄ and LPA₅ was similar in LPA₁ KO and wild type fibroblasts harvested from mice at both time points. See, FIG. 11.

Fibroblast accumulation can be quantified by immunohistochemical staining with a fibroblast specific stain (i.e., for example, anti-fibroblast-specific protein 1 (FSP1) antibody. Lawson et al., “Characterization of Fibroblast Specific Protein 1 in Pulmonary Fibrosis” Am J Respir Crit Care Med 171(8):899-907 (2005). Epub 2004 Dec. 23)). The data presented herein shows that lung cells from both unchallenged wild type and unchallenged LPA₁^{-/-} mice had a minimal response to FSP1 staining. See, FIG. 5d. On Day 14 after a bleomycin challenge, FSP1 staining increased to a greater degree in the wild type mice in comparison with the LPA₁^{-/-} mice. See, FIG. 5d. Specifically, FSP1 staining was 62% lower in LPA₁^{-/-} mice that in the wild type mice (P=0.0017). See, FIG. 5e.

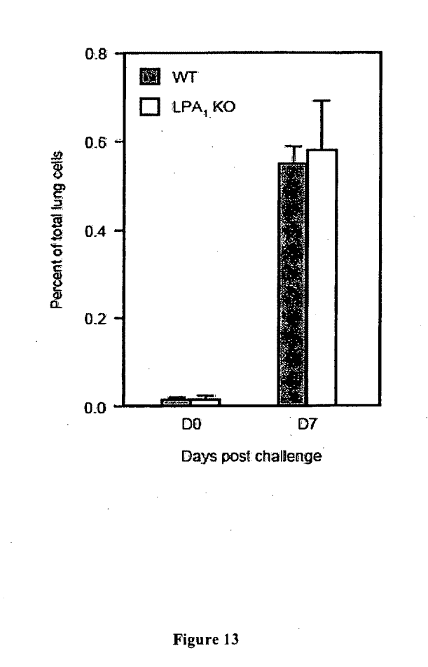

The above observations suggested a determination of whether fibrocyte accumulation was also reduced in the lungs of LPA₁^{-/-} mice following bleomycin administration. Fibrocytes have been identified as circulating fibroblast precursor cells, and have been shown to be recruited to the lungs during the development of pulmonary fibrosis, with peak accumulation occurring approximately one week post-injury in animal models . Phillips et al., “Circulating fibrocytes traffic to the lungs in response to CXCL12 and mediate fibrosis” J Clin Invest 114:438-446 (2004): Bucala et al., “Circulating fibrocytes define a new leukocyte subpopulation that mediates tissue repair” Mol Med 1: 71-81 (1994); and Moore et al., “CCR2-mediated recruitment of fibrocytes to the alveolar space after fibrotic injury” Am J Pathol 166:675-684 (2005). Fibrocytes can be identified using flow cytometry as cells that co-stain with anti-CD45 and anti-collagen type I (Col I) antibodies. Quantities of lung CD45+ Col I+ were compared between LPA₁^{-/-} and wild type mice both before a bleomycin challenge and on Day 7 after a bleomycin challenge. In both LPA₁^{-/-} and wild type mice CD45+ Col I+ cells represented 0.017% of total cells in the lungs before bleomycin challenge. Further, similar responses were seen in both types of mice on Day 7 after bleomycin challenge: 0.55% of lung cells in wild type mice, and 0.58% of lung cells in LPA₁^{-/-} mice. See, FIG. 13.

Although it is not necessary to understand the mechanism of an invention it is believed that the reduced fibroblast accumulation in the lungs of LPA₁-deficient mice is attributable to failure of fibroblasts to respond to the chemoattractant activity generated by injury, consequently suggesting that the generation of the chemoattractant activity itself should be independent of LPA₁. BAL samples were recovered on Day 5 after bleomycin challenge from either wild type mice or LPA₁-deficient (LPA₁^{-/-} mice and tested for their ability to induce chemotaxis of wild type fibroblasts. No significant difference in the fibroblast chemoattractant activity of the BAL from bleomycin-challenged wild type or LPA₁-deficient mice were observed. See, FIG. 5f.

G. LPA₁ Receptors And Fibroblast Proliferation

LPA has been demonstrated to induce fibroblast proliferation as well as migration. Similar to chemotactic activity generation, no significant differences were seen in Day 5 or Day 14 bleomycin-induced BAL sample proliferative responses of wild type and LPA₁-deficient fibroblasts.

Decreased proliferation of LPA₁^{-/-} fibroblasts could contribute to the decreased fibroblast accumulation following a bleomycin challenge. Similarly, a decreased production of collagen by LPA₁-deficient fibroblasts could contribute to the decreased collagen accumulation following a bleomycin. No significant differences, however, were observed between fibroblast proliferative responses of wild type versus LPA₁^{-/-} fibroblasts to BAL samples recovered on Day 5 or Day 14 following a bleomycin challenge. See, FIG. 5g. These data suggest that fibroblast proliferation in response to a bleomycin challenge is independent of LPA₁ receptors.

Further, the expression of the matrix genes procollagen type 1α₁ and fibronectin induced by the pro-fibrotic cytokine TGF-β also was similar in wild type and LPA1-deficient fibroblasts. See, FIG. 5h. These data suggest that fibroblast synthesis of extracellular matrix proteins is independent of LPA₁ receptors. Further, induction of fibroblast α-smooth muscle actin expression by TGF-β was similar in wild type and LPA₁^{-/-} fibroblasts. See, FIG. 5h. Myofibroblasts are a source of collagen type I gene expression in actively fibrosing sites following bleomycin injury. Zhang et al., “Myofibroblasts and their role in lung collagen gene expression during pulmonary fibrosis. A combined immunohistochemical and in situ hybridization study” Am J Pathol 145:114-125 (1994). The unaffected induction of α-smooth muscle actin in LPA₁^{-/-} fibroblasts suggests that the generation of myofibroblasts from fibroblasts is independent of LPA₁ receptors.

Taken together, the above data support the following conclusions: (1) fibroblast accumulation following lung injury is markedly reduced in LPA₁^{-/-} mice; (2) fibroblast migration in response to chemoattractant activity following lung injury is markedly reduced in LPA₁^{-/-} fibroblasts; (3) chemoattractant activity generation following lung injury is not affected in LPA₁^{-/-} mice; (4) fibroblast proliferation following lung injury is not affected in LPA₁^{-/-} mice; and (5) the reduced fibroblast accumulation following lung injury in LPA₁^{-/-} mice explains reduced collagen gene expression and accumulation of collagen protein accumulation rather than intrinsic impairments in collagen synthesis or myofibroblast differentiation. These results confirm previous suggestions in the art that LPA-induced fibroblast proliferation may be mediated by LPA₂. Contos et al., “Characterization of Ipa(2) (Edg4) and Ipa(1)/Ipa(2) (Edg2/Edg4) lysophosphatidic acid receptor knockout mice: signaling deficits without obvious phenotypic abnormality attributable to Ipa(2)” Mol Cell Biol 22:6921-6929 (2002).

In one embodiment, the present invention contemplates a method for inhibiting lung fibroblast accumulation using an LPA₁ inhibitor. In one embodiment, the LPA₁ inhibitor reduces fibroblast migration. In one embodiment, the fibroblast migration is induced by a chemoattractant induced by lung injury. In one embodiment, the chemoattractant is LPA.

G. LPA₁ Receptors And Vascular Leak

It has been reported that LPA may increase vascular permeability. van Nieuw Amerongen et al., “Role of RhoA and Rho kinase in lysophosphatidic acid-induced endothelial barrier dysfunction” Arterioscler Thromb Basc Biol 20:E127-E133 (2000); and Neidlinger et al., “Hydrolysis of phosphatidylserine-exposing red blood cells by secretory phospholipase A2 generates lysophosphatidic acid and results in vascular dysfunction” J Biol Chem 281:775-781 (2006). In one embodiment, the present invention contemplates a method for reducing vascular leak by administering a LPA₁ receptor inhibitor.

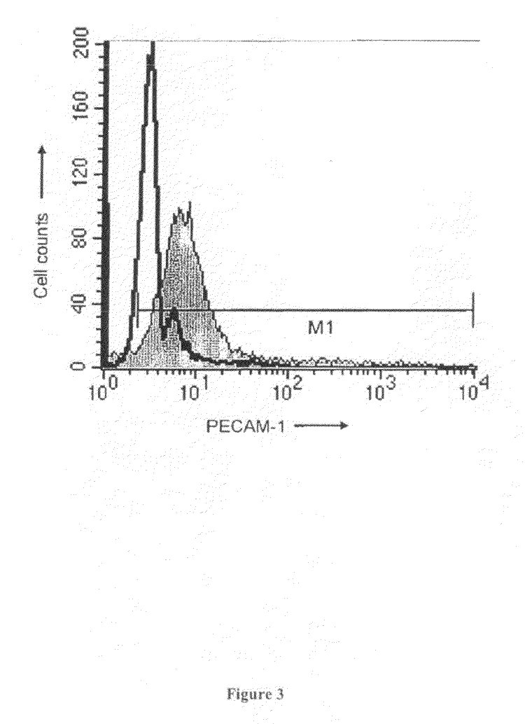

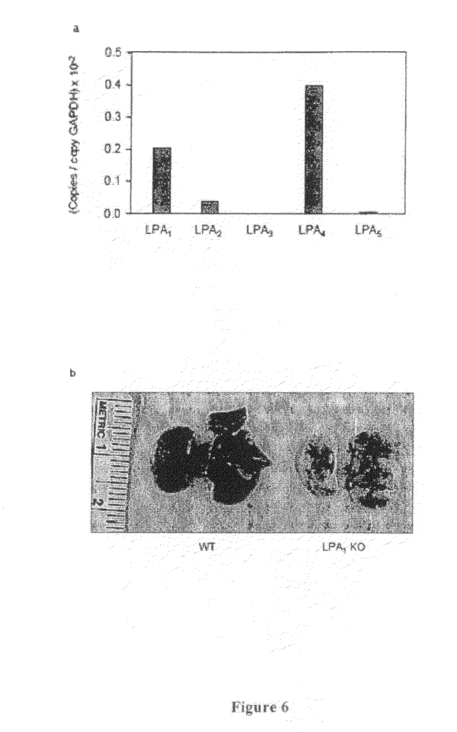

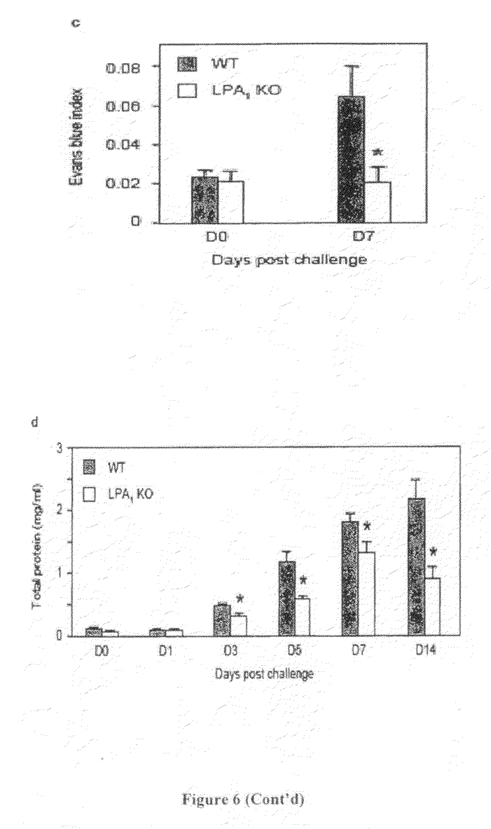

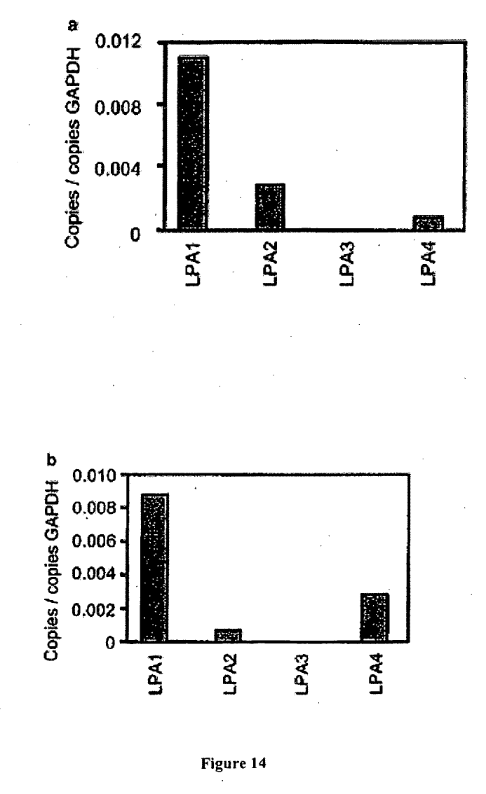

Alternatively, LPA-induced vascular leak may be reduced by reducing LPA₁ receptor expression. The data presented herein explores endothelial cell LPA receptor expression measured by Quantitative Polymerase Chain Reaction (QPCR). LPA₁ was observed to be a highly expressed LPA receptor in both mouse C166 yolk-sac-derived endothelial cell line and by mouse primary cardiac endothelial cells. See, FIG. 14a and FIG. 14b, respectively. LPA receptor expression of primary endothelial cells isolated from mouse lungs were shown to predominantly express LPA₁ and LPA₄. See, FIG. 6a. The nature of the endothelial cells were confirmed by showing PECAM-1 expression of greater than 90%. See, FIG. 3.

Vascular leak induced by bleomycin injury in LPA₁^{-/-} mice as compared to wild type was determined by Evans blue dye extravasation and BAL total protein concentration in these mice following bleomycin challenge. The data presented herein shows increased vascular leak from wild type bleomycin-injured lung parenchyma as compared to LPA₁^{-/-} bleomycin-injured lung parenchyma. For example, dramatically more Evans blue dye extravasated from the vasculature into the lung parenchyma of bleomycin-injured wild type mice compared to LPA₁-deficient mice. On Day 7, Evans blue dye extravasation was significantly increased (as seen by the deep purple lung tissue) in wild type mice challenged with bleomycin as compared to unchallenged wild type mice. See, FIG. 6b (left). In contrast, on Day 7 Evans blue dye extravasation was minimally increased (as seen by the pale blue lung tissue) in LPA₁^{-/-} deficient mice challenged with bleomycin as compared to unchallenged LPA1^{-/-} mice. See, FIG. 6b (right). A quantification of the Evans blue dye index (see Examples) shows that the wild type mice have significantly more extravasation than the LPA₁^{-/-} mice. See, FIG. 6c. Elevated total protein concentration normally observed in bleomycin-induced BAL samples from wild type mice were significantly reduced in LPA₁^{-/-} (mice on Day 3 , Day 5, Day 7 and Day 14 after bleomycin administration. For example, protein was elevated by 36% on Day 3 after bleomycin challenge (P=0.022), and by 51% on Day 5 after bleomycin challenge (P=0.014). See, FIG. 6d.

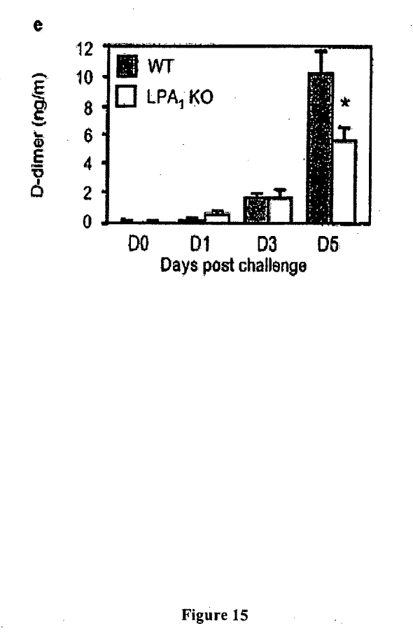

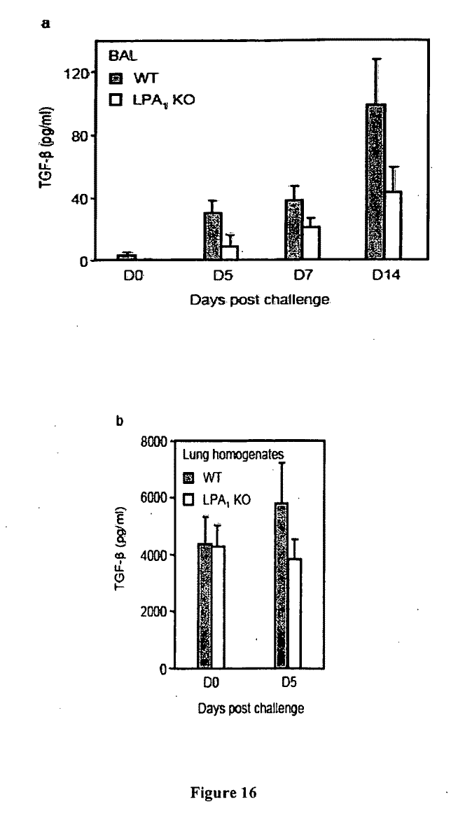

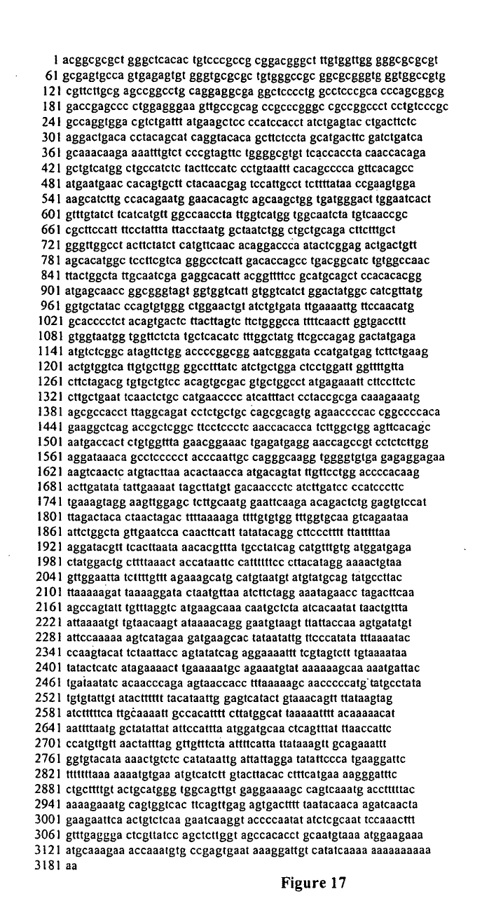

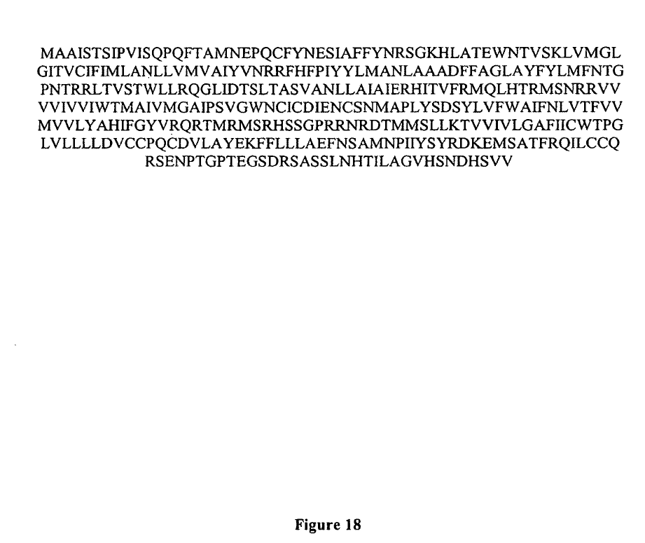

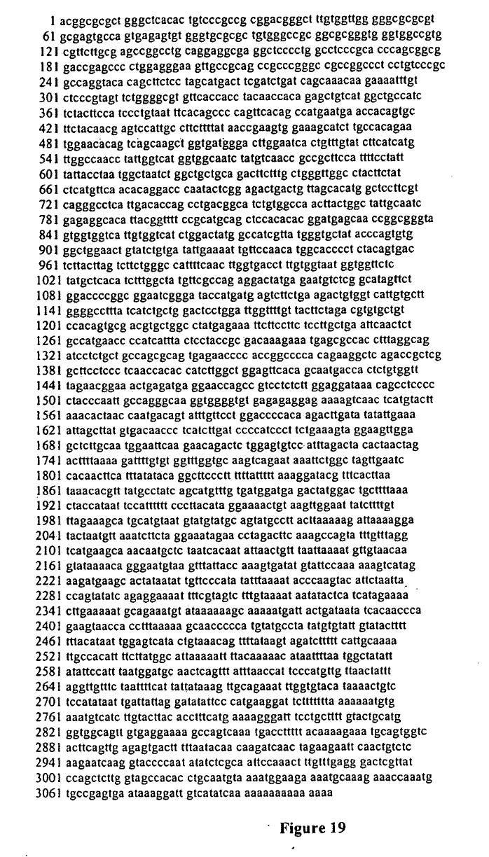

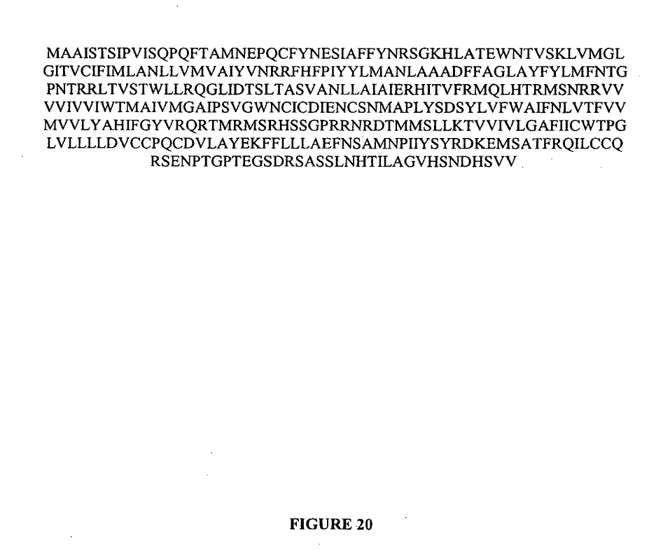

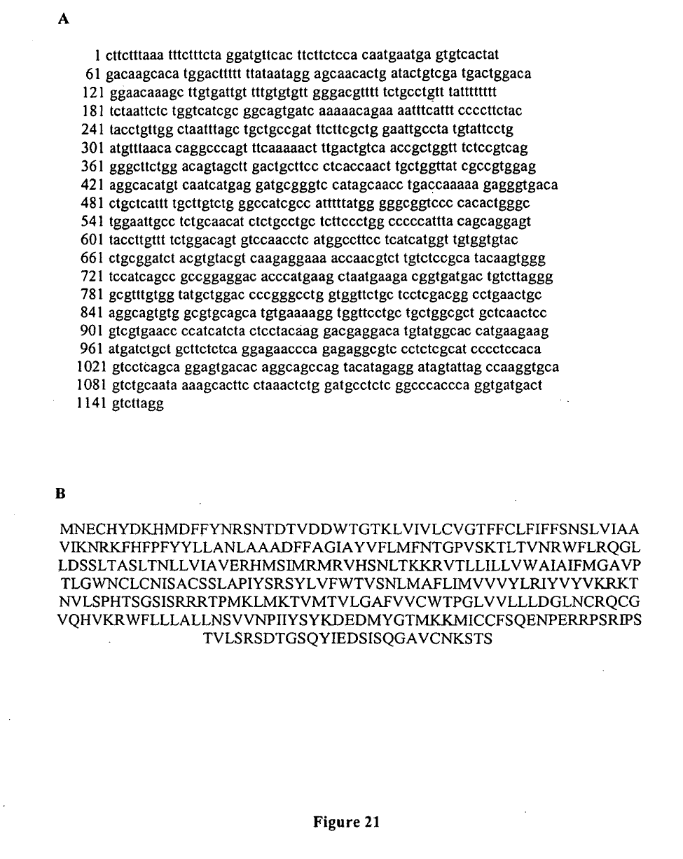

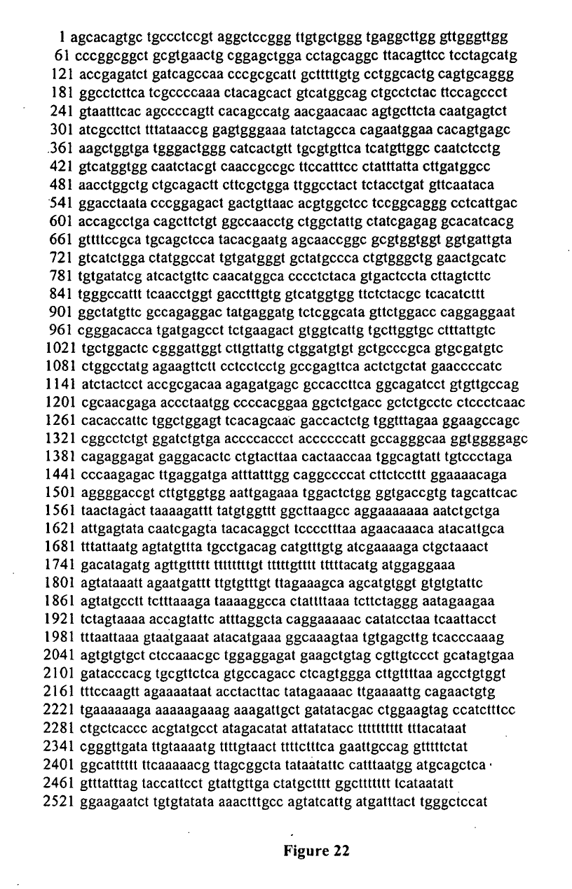

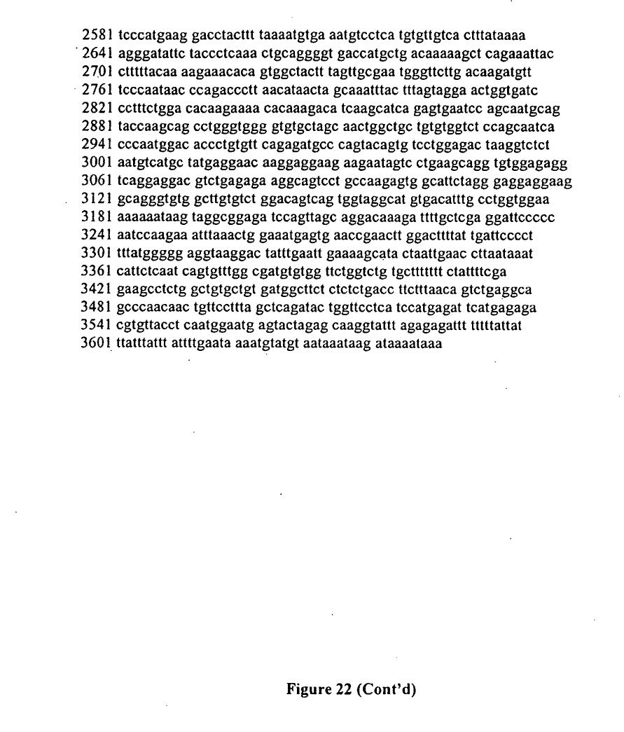

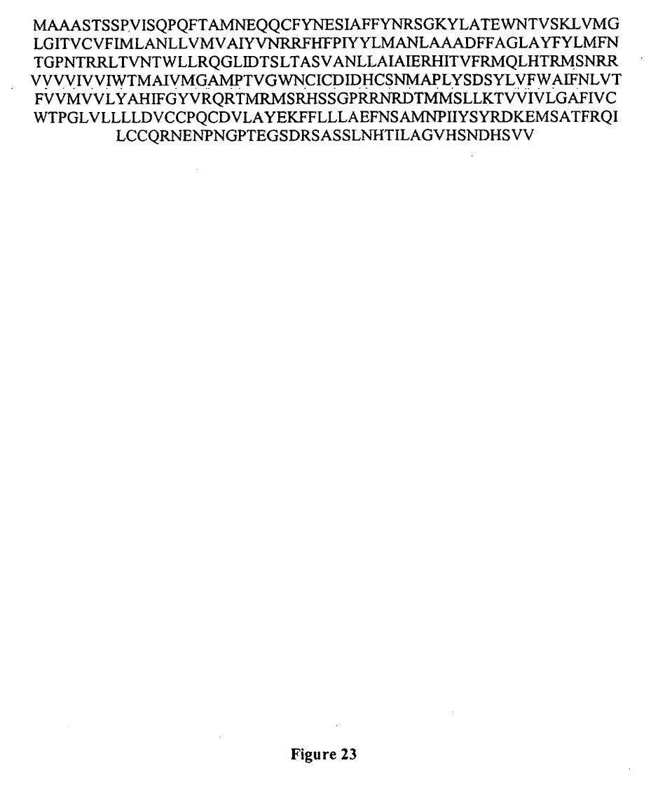

Further, fibrin deposition resulting from vascular leak is thought to contribute to the development of fibrosis induced by lung injury. Chambers et al., “Coagulation cascade proteases and tissue fibrosis” Biochem Soc Trans 30: 194-200 (2002). It has been reported that elevations of BAL D-dimer, a proteolytic fragment of fibrin that has been stabilized by Factor XIII during coagulation, correlate with fibrotic lung disease severity, including idiopathic pulmonary fibrosis (IPF). Gunther et al., “Enhanced tissue factor pathway activity and fibrin turnover in the alveolar compartment of patients with interstitial lung disease” Thromb Haemost 83:853-860 (2000). Although it is not necessary to understand the mechanism of an invention it is believed that reduced vascular leakage in LPA₁^{-/-} mice is reflected by reduced intra-alveolar fibrin deposition and turnover following lung injury. The data presented herein shows that Day 5 bleomycin-induced BAL sample D-dimer concentrations were reduced by 45% in LPA₁^{-/-} mice as compared with wild type mice (P=0.0087). See, FIG. 15. In one embodiment, the present invention contemplates a method for inhibiting fibrin deposition by administering an LPA₁ receptor inhibitor.

H. LPA₁ Receptors And Leukocyte Recruitment

LPA may also be a leukocyte chemoattractant. Although it is not necessary to understand the mechanism of an invention it is believed that if LPA contributes to leukocyte recruitment following bleomycin injury, then reduced leukocyte accumulation in the lungs of LPA₁-deficient mice could also provide protection from bleomycin-induced fibrosis.

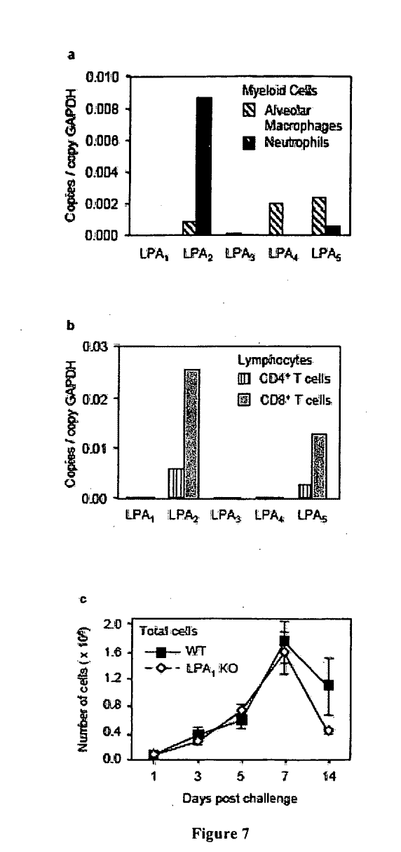

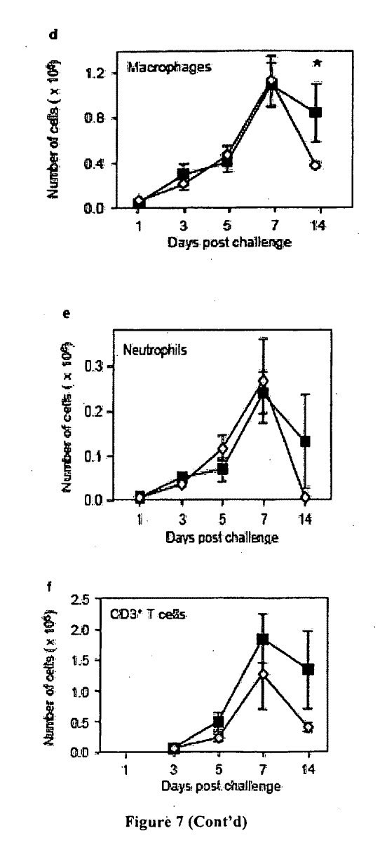

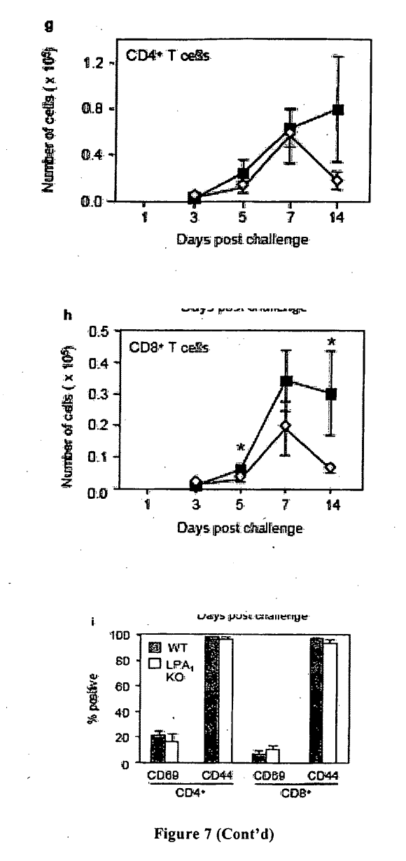

LPA receptor expression was measured in leukocyte subsets recruited into the lung airspaces following bleomycin administration in mice. Alveolar macrophages, neutrophils, and lymphoid (CD4⁺ and CD8⁺) lineages expressed little or no LPA₁ receptors, but did express substantial amounts of other LPA receptor subtypes (i.e., for example, LPA₂). See, FIG. 7a and FIG. 7b, respectively.

Further, no significant differences between the numbers of total leukocytes, macrophages, or neutrophils present in bleomycin-induced BAL samples from LPA₁^{-/-} and wild type mice at Day 1, Day 3, Day 5, and Day 7. See, FIG. 7c, FIG. 7d, and FIG. 7e, respectively. Similarly, no significant differences were observed when comparing the numbers of CD3⁺ T cells, CD4⁺ T cells, or CD8⁺ T cells present in bleomycin-induced BAL samples from LPA₁^{-/-}and wild type mice at Day 3, Day 5, and Day 7. See, FIG. 7f, FIG. 7g, and FIG. 7h, respectively. Leukocyte recruitment ultimately dropped off in LPA₁^{-/-} mice on Day 14 following bleomycin administration, presumably because of a faster resolution of repair processes in an environment of diminished fibrosis. These data indicate that leukocyte recruitment following bleomycin challenge occurs independently of LPA₁ receptors.

The functional phenotype and activation status of lymphocytes recruited to the lungs of LPA₁-deficient and wild type mice following bleomycin challenge. On Day 5 following bleomycin administration, almost all CD4+ and CD8+ T cells recovered in BAL from both LPA₁^{-/-} and wild type mice had a memory phenotype. See, FIG. 7i. Memory phenotype is usually indicated by CD44 high expression. Picker et al., “Differential expression of homing-associated adhesion molecules by T cell subsets in man” J Immunol 145:3247-3255 (1990). The percentages BAL CD4+ and CD8+ T cells that had recently been activated, as indicated by CD69 expression were also similar in LPA₁-deficient and wild type mice. Hara et al., “Human T cell activation. III. Rapid induction of a phosphorylated 28 kD/32 kD disulfide-linked early activation antigen (EA 1) by 12-o-tetradecanoyl phorbol-13-acetate, mitogens, and antigens” J Exp Med 164:1988-2005 (1986); and Maino et al., “Rapid flow cytometric method for measuring lymphocyte subset activation” Cytometry 20:127-133 (1995). See, FIG. 7i. These data indicate that leukocyte recruitment and activation following bleomycin challenge initially occur independently of LPA₁ receptors, and that the inflammatory and fibrotic responses to lung injury are uncoupled in the absence of LPA₁ expression.

In one embodiment, the present invention contemplates that LPA signaling generated during a lung injury is mediated by an LPA1 receptor, thereby resulting in both vascular leak and fibroblast recruitment. In one embodiment, vascular leak and fibroblast recruitment occur sequentially. In one embodiment, vascular leak and fibroblast recruitment act together during the development of injured tissue fibrosis. In one embodiment, LPA₁ receptors mediate persistant vascular leak, thereby leading to excessive deposition of fibrin. Although it is not necessary to understand the mechanism of an invention it is believed that extravascular fibrin provides a provisional matrix for fibroblast invasion subsequent to LPA-induced recruitment. The lack of pulmonary fibrosis development in LPA₁^{-/-} mice after a bleomycin challenge is most likely due to a concurrent mitigation of LPA-mediated vascular leak and fibroblast recruitment. In some embodiments, the present invention contemplates that LPA₁ receptors comprise a target to treat fibrotic diseases. In one embodiment, the diseases are refractory to currently available treatments. In one embodiment, the diseases are selected from the group comprising IPF or ARDS.

### IV. Lysophosphatidic Acid Inhibitors

LPA activity may also be regulated by protein binding that either sequesters LPA or improved receptor delivery. Goetzl et al., “Pleiotypic mechanisms of cellular responses to biologically active lysophospholipids” Prostaglandins Other Lipid Mediat 64:11-20 (2001). As demonstrated herein using fibroblast chemotaxis, some LPA activities may be potentiated by albumin binding. For example, a protein that can inhibit LPA activity is plasma gelsolin, which binds LPA with an affinity considerably greater than that of albumin. Goetzl et al., “Gelsolin binding and cellular presentation of lysophosphatidic acid” J Biol Chem 275:14573-14578 (2000). Plasma gelsolin levels decline significantly after tissue injury, and the resultant increased activity of LPA due to gelsolin depletion in plasma and tissue fluids may exacerbate post-injury organ dysfunction. Dinubile M. J., “Plasma gelsolin: in search of its raison d'etre” Am J Physiol 292:C1240-1242 (2007). Although it is not necessary to understand the mechanism of an invention, it is believed that increased fibroblast chemotactic activity of BAL samples from bleomycin-injured mice and/or IPF patients may result from increased albumin levels, in addition to decreased levels of LPA inhibitory proteins. It has been reported that 11³F BAL samples contain higher albumin levels. Jones et al., “A comparison of albumin and urea as reference markers in bronchoalveolar lavage fluid from patients with interstitial lung disease” Eur Respir J 3:152-156 (1990).

A. LPA Receptors And Fibroblast Proliferation

Azole compounds have been reported to modify the physiological activity of lysophosphatidic acid by an LPA receptor antagonistic action. Yamamoto et al. “Novel azole compound”, United States Patent Application Number 2006/0194850 (filed Feb. 3, 2006). These azole compounds were not differentiated as to whether the compounds are specific for LPA₁, LPA₂ or both, but were reported to stimulate fibroblast lung cell proliferation during the development of fibrosis. As a result, these azole LPA receptor antagonists were suggested to be agents for the prophylaxis or treatment of fibroblast cell proliferation during lung fibrosis. Yamamoto et al., however, provided no data demonstrating that any LPA receptor antagonist was actually effective treating fibroblast proliferative-related disorders, much less pulmonary fibrosis. Surprisingly, however, the data presented herein has demonstrated that a lung fibroblast proliferative response induced by LPA is not mediated by LPA₁ receptors. Further, Yamamoto et al. does not teach any LPA antagonists for reducing fibrin deposition. Especially, LPA-induced fibrin deposition observed during the development of lung fibrosis.

Lysophosphatidic acid analogs have been used as agonists or antagonists of LPA₁, LPA₂, and LPA₃ receptors. Lynch et al. “Lysophosphatidic acid receptor agonists and antagonists”, U.S. Pat. No. 7,169,818. These analog LPA compounds are 2-substituted ethanolamide derivatives exhibiting differing degrees of size, hydrophobicity, and stereochemistry. The parent N-acyl ethanolamine phosphate is nearly indistinguishable from LPA in stereochemical interactions with both the LPA₁ and the LPA₂ receptors. Further, some LPA antagonists have different affinities (and therefore efficacies) between the three different receptor subtypes. These LPA analogs are suggested for the treatment of diseases characterized by cell hyperproliferation. Surprisingly, however, the data presented herein has demonstrated that lung fibroblast proliferative responses induced by LPA is not mediated by LPA₁ receptors. Further, Lynch et al. does not teach any LPA antagonists for reducing fibrin deposition. Especially, LPA-induced fibrin deposition observed during the development of lung fibrosis.

LPA₁ and LPA₂ regulation has been suggested to inhibit the development of nasal polyps, a form of cellular hyperproliferation. Barekzi et al. (2006) Lysophosphatidic acid stimulates inflammatory cascade in airway-epithelial cells, Prostaglandins, Leukotrienes, and Essential Fatty Acids 74:357-363. The LPA₁ and LPA₂ receptors were observed to be constitutively expressed in lung and nasal polyp-derived epithelial cells when exposed to LPA. It was suggested that cellular hyperproliferation resulting from LPA₁ and LPA₂ mRNA expression may be mediated by a variety of signaling cytochemicals. The data presented herein, however, has demonstrated that lung fibroblast proliferative responses induced by LPA is not mediated by LPA₁ receptors. Further, Barekzi et al. does not teach any LPA antagonists for reducing fibrin deposition. Especially, LPA-induced fibrin deposition observed during the development of lung fibrosis.

Farnesyl phosphates are proposed regulators of LPA receptor activity. Liliom et al. (2006) Farnesyl phosphates are endogenous ligands of lysophosphatidic acid receptors: Inhibition of LPA GPCR and activation of PPARs, Biochimica et Biophysica Acta 1761, 1506-1514. Farnesyl phosphates may regulate LPA receptors in pathways relevant to steroid synthesis and the posttranslational isoprenylation of proteins.

Potential links between LPA-specific receptors and apoptotic pathways related to brain and retinal ischemia have been reported. Savitz et al. (2006) EDG receptors as a potential target in retinal ischemia-reperfusion injury, Brain Research 1118, 168-175. An analog of LPA may be involved in an upregulation of LPA receptors in response to ischemia. It was concluded that LPA receptors may provide neuroprotection in retinal ischemia-reperfusion injury.

A potential role for an LPA receptor antagonist in controlling fibroblast differentiation into myofibroblasts, a phenomenon associated with the onset of breast cancer has been reported. Yin et al. (2005) “Chloride channel activity in human lung fibroblasts and myofibroblasts” American Journal of Physiology—Lung, Cellular and Molecular Physiology 288, 1110-1116. Elevated levels of dioctyl-glycerol pyrophosphate, an LPA receptor-specific antagonist, blocked the activation of an LPA-activated chloride channel thought to be involved in human lung fibroblast differentiation.

B. LPA₁ Receptor Inhibitors And Fibroblast Chemotaxis

The extent to which fibroblast chemotaxis induced by post-injury BAL samples was attributable to LPA was determined measuring the extent to which this chemotaxis was reduced when fibroblast LPA₁ function was inhibited with chemical antagonists, or when fibroblast LPA₁ expression was absent in cells isolated from LPA₁^{-/-} mice.

1. Small Organic Molecule Inhibitors

The LPA receptor antagonist Ki16425 inhibits LPA-induced responses mediated by LPA₁≧LPA₃>>LPA₂, without appreciable effects on cellular responses mediated by closely related lipid receptors, such as sphingosine 1-phosphate receptors. Ohta et al., “Ki16425, a subtype-selective antagonist for EDG-family lysophosphatidic acid receptors” Mol Pharmacol 64: 994-1005 (2003). The data presented herein shows that Ki16425 significantly inhibited fibroblast chemotaxis induced by BAL samples from mice on Day 5 after a bleomycin challenge. Ki16425, however, did not affect PDGF-induced fibroblast chemotaxis. See, FIG. 5b.

VPC12249, is another specific LPA antagonist that inhibits LPA-induced responses mediated by LPA₁≧LPA₃>>LPA₂. Heise et al., “Activity of 2-substituted lysophosphatidic acid (LPA) analogs at LPA receptors: discovery of a LPA1/LPA3 receptor antagonist” Mol Pharmacol 60:1173-1180 (2001). The data presented herein shows that VPC12249 inhibited fibroblast chemotaxis induced by BAL samples from mice 5 Days after a bleomycin challenge. PDGF induced fibroblast chemotaxis was not affected by VPC12249. See, FIG. 10.

2. Antisense Inhibitors

In one embodiment, the present invention contemplates a nucleic acid comprising a sequence at least partially complementary with the LPA₁ receptor DNA coding region. In one embodiment, the nucleic acid comprises an antisense sequence.

In one embodiment, the present invention contemplates a method comprising administering an antisense sequence to a fibrosis patient under conditions such that the LPA₁ receptor population is reduced. In one embodiment, the antisense sequence is at least partially complementary to at least a portion of an LPA₁ receptor DNA. In one embodiment, the antisense sequence is partially complementary to at least a portion of an LPA₁ receptor mRNA. In one embodiment, the mRNA comprises SEQ ID NO:1.

Although it is not necessary to understand the mechanism of an invention it is believed an LPA1 antisense inhibitor will inhibit LPA-induced fibroblast chemotaxis. It is further believed that an LPA₁ antisense inhibitor will reduce the development of fibrosis.

In some embodiments, detection of LPA₁ receptor expression comprises measuring the expression of corresponding mRNA in a biological sample (i.e., for example, a blood sample). mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In other embodiments, RNA expression is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific nucleic acid (e.g., RNA) sequences by using structure-specific enzymes to cleave a complex formed by the hybridization of overlapping oligonucleotide probes.

In still further embodiments, RNA (or corresponding cDNA) is detected by hybridization to an oligonucleotide probe. A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In other embodiments, LPA₁ receptor gene expression may be detected by measuring the expression of a protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding may be detected by many different techniques including, but not limited to, (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized. In other embodiments, the immunoassay described in U.S. Pat. Nos. 5,599,677 and 5,672,480; each of which is herein incorporated by reference.

In some embodiments, a computer-based analysis program is used to translate the raw data generated by the detection assay (e.g., the presence, absence, or amount of a given marker or markers) into data of predictive value for a clinician. A clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, wherein the information is provided to medical personal and/or subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of a virus infection) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

3. Protein Inhibitors

a. Peptides

In one embodiment, the present invention contemplates a peptide capable of attaching to an LPA receptor under conditions such that fibrosis development is reduced. In one embodiment, the peptide comprises the reverse amino acid sequence of a portion of the LPA receptor. In one embodiment, the portion encodes a binding pocket of the LPA receptor. In one embodiment, the LPA receptor comprises an LPA₁ receptor.

b. Antibodies

The present invention provides isolated antibodies (i.e., for example, polyclonal or monoclonal). In one embodiment, the present invention provides monoclonal antibodies that specifically bind to an isolated polypeptide comprised of at least five amino acid residues of the receptor proteins described herein (e.g., LPA₁). These antibodies find use in the treatment methods described above.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO2 gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a protein expressed on a fibroblast and/or leukocyte (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### V. Human Fibrotic Lung Disease

In one embodiment, the present invention contemplates a method providing a patient having idiopathic pulmonary fibrosis and detecting increased lysophosphatidic acid levels in bronchoalveolar lavage fluid derived from the patient.

In one embodiment, the present invention contemplates a composition comprising a bronchoalveolar lavage sample, wherein the sample comprises fibroblast chemotactic activity. In one embodiment, the sample is derived from an idiopathic pulmonary fibrosis patient. In one embodiment, the fibroblast chemotactic activity is lysophosphatidic acid.

In one embodiment, the present invention contemplates a method providing a patient having idiopathic pulmonary fibrosis, wherein the fibrosis comprises elevated fibroblast chemotactic activity and administering an LP/⁶i_{′} receptor inhibitor under conditions such that the fibroblast chemotactic activity is reduced. In one embodiment, the fibroblast chemotactic activity is detected in a bronchoalveolar lavage sample.

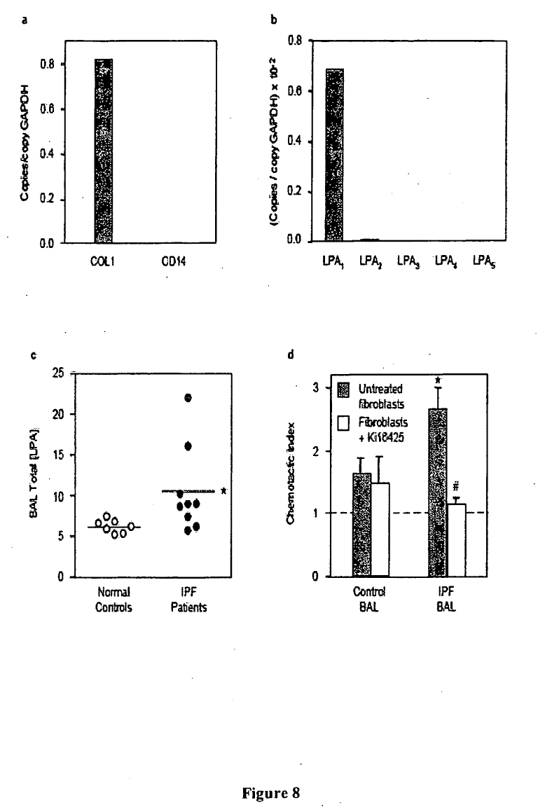

The role of the LPA-LPA₁ pathway in fibroblast migration was investigated using patients having idiotypic pulmonary fibrosis, a prototypic human fibrotic lung disease. Fibroblast LPA receptor expression present in IPF BAL fluid are presented by fibroblasts that have migrated into the lung airspaces (i.e., for example, alveoli), but such fibroblast migration does not occur in non-diseased patients. Fireman et al., “Morphological and biochemical properties of alveolar fibroblasts in interstitial lung diseases” Lung 179:105-117 (2001). Following centrifugation, pelleted BAL fibroblast cells were grown in culture for multiple passages, and had the morphologic appearance of fibroblasts. These cultured cells were confirmed as fibroblasts because the relative expression of collagen type Iα₁ (characteristic of fibroblasts) was much greater than that of CD 14 (characteristic of macrophages). See FIG. 8a. The data presented herein shows that the LPA₁ receptor was the most highly expressed LPA receptor on human IPF fibroblasts. See, FIG. 8b.

As determined by ESI-MS, total LPA concentrations in human IPF BAL samples were significantly higher than concentrations in BAL from normal controls (i.e., for example, non-IPF humans). See, FIG. 8c. These data are consistent with previous observations that fibroblast migration chemoattractants recovered in IPF BAL samples are absent in control subjects, and that a positive correlation exists between the magnitude of IPF BAL fibroblast chemoattractant activity and IPF severity and progression. Behr et al., “Fibroblast chemotactic response elicited by native bronchoalveolar lavage fluid from patients with fibrosing alveolitis” Thorax 48:736-742 (1993); and Selman et al., “Accelerated variant of idiopathic pulmonary fibrosis: clinical behavior and gene expression pattern” PLoS ONE 2 e482 (2007).

Fibroblast chemotaxis experiments were performed using human fetal lung fibroblasts (HFL1 cells) showing that human IPF BAL samples induced significantly greater fibroblast chemotaxis than non-IPF BAL samples. See, FIG. 8d. Further, the LPA₁ antagonist Ki16425 significantly inhibited fibroblast chemotaxis induced by IPF BAL samples, indicating that fibroblast migration induced by human IPF BAL samples is mediated by LPA₁ receptors. See, FIG. 8d.

### VI. Screening Techniques

In some embodiments, the present invention provides drug screening assays (e.g., to screen for LPA₁ inhibitor drugs). Specifically, the present invention provides screening methods for identifying modulators, i.e., candidate or test compounds or agents (e.g., proteins, peptides, peptidomimetics, peptoids, small molecules or other drugs) which bind to an LPA receptor and have an inhibitory (or stimulatory) effect on, for example, fibroblast migration and/or recruitment. Compounds thus identified can be used to prevent fibrosis by using them either directly or indirectly in a therapeutic protocol Compounds which inhibit LPA receptors are useful in the treatment of fibrosis.

In one embodiment, the invention provides assays for screening candidate or test compounds that are substrates of an LPA receptor or a portion thereof. In another embodiment, the invention provides assays for screening candidate or test compounds that bind to or modulate the activity of an LPA receptor or portion thereof The test compounds of the present invention can be obtained using any of the numerous approaches in combinatorial library methods, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678 85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Antivirus induced Drug Des. 12:145).

Numerous examples of methods for the synthesis of molecular libraries have been reported, for example in: DeWitt et al., Proc. Natl. Acad. Sci. U.S.A. 90:6909 [1993]; Erb et al., Proc. Natl. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412 421 [1992]), or on beads (Lam, Nature 354:82 84 [1991]), chips (Fodor, Nature 364:555 556 [1993]), bacteria or spores (U.S. Pat. No. 5,223,409; herein incorporated by reference), plasmids (Cull et al., Proc. Natl. Acad. Sci. USA 89:18651869 [1992]) or on phage (Scott and Smith, Science 249:386 390 [1990]; Devlin Science 249:404 406 [1990]; Cwirla et al., Proc. Natl. Acad. Sci. 87:6378 6382 [1990]; Felici, J. Mol. Biol. 222:301 [1991]).

In one embodiment, an assay is a cell-based assay in which a cell that expresses an LPA receptor or portion thereof is contacted with a test compound, and the ability of the test compound to inhibit fibrosis is determined. Determining the ability of the test compound to modulate fibrosis development can be accomplished by monitoring, for example, changes in pulmonary function. The cell, for example, can be of mammalian origin.

The ability of the test compound to modulate LPA receptor binding to an inhibitory compound, can also be evaluated. This can be accomplished, for example, by coupling the compound, e.g., the substrate, with a radioisotope or enzymatic label such that binding of the compound, e.g., the substrate, can be determined by detecting the labeled compound, e.g., substrate, in a complex.

Alternatively, the LPA receptor is coupled with a radioisotope or enzymatic label to monitor the ability of a test compound to binding to the substrate. For example, compounds (e.g., substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with an LPA receptor with or without the labeling of any of the interactants can be evaluated. For example, a microphysiometer can be used to detect the interaction of a compound with an LPA receptor without the labeling of either the compound or the receptor (McConnell et al. Science 257:1906 1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and receptors.

In yet another embodiment, a cell-free assay is provided in which an LPA receptor or portion thereof is contacted with a test compound and the ability of the test compound to bind to the receptor or portion thereof is evaluated. In one embodiment, a portion of the LPA receptor to be used in assays of the present invention include fragments that participate in interactions with substrates or other proteins, e.g., fragments with high surface probability scores. Cell-free assays involve preparing a reaction mixture of the receptor and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter).

In another embodiment, determining the ability of an LPA receptor to bind to a test compound can be accomplished using real-time Biomolecular Interaction Analysis (BIA) (see, e.g., Sjolander and Urbaniczky, Anal. Chem. 63:2338 2345 [1991] and Szabo et al. Curr. Opin. Struct. Biol. 5:699 705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the LPA receptor or the test compound is anchored onto a solid phase. These complexes are anchored on the solid phase can be detected at the end of the reaction. Preferably, the receptor can be anchored onto a solid surface, and the test compound, (which is not anchored), can be labeled, either directly or indirectly, with detectable labels discussed herein.

Alternatively, the complexes can be dissociated from the matrix, and the level of test compound binding to the receptor determined using standard techniques. Other techniques for immobilizing either receptor proteins or a target compound on matrices include using conjugation of biotin and streptavidin. Biotinylated virus induced marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with LPA receptors or test compounds which do not interfere with binding of the receptor and test compound. Such antibodies can be derivatized to the wells of the plate, and unbound target or virus induced markers protein trapped in the wells by antibody conjugation. Methods for detecting such complexes, in addition to those described above for the GST-immobilized complexes, include immunodetection of complexes using antibodies reactive with the receptor or test compound, as well as enzyme-linked assays which rely on detecting an enzymatic activity associated with the receptor or test compound.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284 7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11: 141 8 [1998]; Hageand Tweed J. Chromatogr. Biomed. Sci. Appl 699:499 525 [1997]). Further, fluorescence energy transfer may also be conveniently utilized, as described herein, to detect binding without further purification of the complex from solution.

The assay can include contacting the receptor proteins or portion thereof with a known compound that binds the receptor to form an assay mixture, contacting the assay mixture with a test compound, and determining the ability of the test compound to interact with a marker protein, wherein determining the ability of the test compound to interact with a marker protein includes determining the ability of the test compound to preferentially bind to markers or biologically active portion thereof, or to modulate the activity of a receptor, as compared to the known compound.

This invention further pertains to novel agents identified by the above-described screening assays. Accordingly, it is within the scope of this invention to further use an agent identified as described herein (e.g., and LPA₁ receptor inhibitory agent, an LPA₁ specific antibody, or an LPA₁ marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### VII. Pharmaceutical Formulations

The present invention further provides pharmaceutical compositions (e.g., comprising the small organic compounds and/or antibody compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VIII. Clinical Administration

The present invention contemplates several drug delivery systems that provide for roughly uniform distribution and have controllable rates of compound release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In one embodiment, the present invention contemplates a medical device comprising several components including, but not limited to, a reservoir comprising an LPA₁ receptor inhibitor, a catheter, a sprayer or a tube. In one embodiment, said medical device administers either an internal or external spray to a patient. In another embodiment, said medical device administers either an internal or external gel to a patient.

Microparticles

In one embodiment, the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Liposomes

In one embodiment, the present invention contemplates liposomes capable of attaching and releasing LPA₁ receptor inhibitor compounds. Liposomes are microscopic spherical lipid bilayers surrounding an aqueous core that are made from amphiphilic molecules such as phospholipids. For example, one liposome embodiment comprises an inhibitor compound trapped between hydrophobic tails of a phospholipid micelle. Water soluble drugs can be entrapped in the core and lipid-soluble drugs can be dissolved in the shell-like bilayer. Liposomes have a special characteristic in that they enable water soluble and water insoluble chemicals to be used together in a medium without the use of surfactants or other emulsifiers.

Liposomes may form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In one embodiment, the present invention contemplates cationic and anionic liposomes, as well as liposomes having neutral lipids comprising an LPA receptor inhibitor. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

In one embodiment, the present invention contemplates a medium comprising liposomes that provide controlled release of LPA₁ inhibitor compounds. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids.

Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel. The terms “microspheres, microcapsules and microparticles” (i.e., measured in terms of micrometers) are synonymous with their respective counterparts “nanospheres, nanocapsules and nanoparticles” (i.e., measured in terms of nanometers). The terms “micro/nanosphere, micro/nanocapsule and micro/nanoparticle” are used interchangeably, as will the discussion herein.

Microspheres are obtainable commercially (Prolease®, Alkerme's: Cambridge, Mass.). For example, a freeze dried LPA receptor inhibitor medium is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of LPA₁ receptor inhibitor release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed solution of an LPA₁ receptor inhibitor is added to the biodegradable polymer metal salt solution. The weight ratio of an LPA receptor inhibitor to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and inhibitor is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and inhibitor mixture are: i) phase separation during a gradual addition of a coacervating agent; ii) an in-water drying method or phase separation method, where an antiflocculant is added to prevent particle agglomeration and iii) by a spray-drying method. In one embodiment, the present invention contemplates a medium comprising a microsphere or microcapsule capable of delivering a controlled release of a compound for a duration of approximately between 1 day and 6 months.

Controlled release microcapsules may be produced by using known encapsulation techniques such as centrifugal extrusion, pan coating and air suspension. Using techniques well known in the state of the art, these microspheres/microcapsules can be engineered to achieve particular release rates. For example, Oliosphere® (Macromed) is a controlled release microsphere system. These particular microsphere's are available in uniform sizes ranging between 5-500 μm and composed of biocompatible and biodegradable polymers. It is well known in the art that specific polymer compositions of a microsphere control the drug release rate such that custom-designed microspheres are possible, including effective management of the burst effect. ProMaxx® (Epic Therapeutics, Inc.) is a protein-matrix drug delivery system. The system is aqueous in nature and is adaptable to standard pharmaceutical drug delivery models. In particular, ProMaxx® are bioerodible protein microspheres that deliver both small and macromolecular drugs, and may be customized regarding both microsphere size and desired drug release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragi® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, a microparticle contemplated by this invention comprises a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005% -0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a compound (i.e., for example, an LPA₁ receptor inhibitor) is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

### IX. Kits

In some embodiments, the present invention provides kits for the detection and characterization of LPA₁ receptor nucleic acids and/or proteins. In some embodiments, the kits contain antibodies specific for a protein expressed as a result of a virus infection, in addition to detection reagents and buffers. In other embodiments, the kits contain reagents specific for the detection of mRNA or cDNA (e.g., oligonucleotide probes or primers). In some embodiments, the kits contain all of the components necessary to perform a detection assay, including all controls, directions for performing assays, and any necessary software for analysis and presentation of results.

In another embodiment, the present invention contemplates kits for the practice of the methods of this invention. The kits preferably include one or more containers containing a DNA detection method of this invention. The kit can optionally include a normal cell culture to be utilized as a control (i.e., for example, a fibroblast cell culture). The kit can optionally include an LPA₁ receptor inhibitor as contemplated herein. The kit can optionally include nucleic acids capable of hybridizing to an LPA₁ gene region (i.e., for example, PCR primers and/or antisense sequences). The kit can optionally include enzymes capable of performing PCR (i.e., for example, DNA polymerase, Taq polymerase and/or restriction enzymes). The kit can optionally include a pharmaceutically acceptable excipient and/or a delivery vehicle (e.g., a liposome). The reagents may be provided suspended in the excipient and/or delivery vehicle or may be provided as a separate component which can be later combined with the excipient and/or delivery vehicle. The kit may optionally contain additional therapeutics to be co-administered with the LPA₁ receptor inhibitor.

The kits may also optionally include appropriate systems (e.g. opaque containers) or stabilizers (e.g. antioxidants) to prevent degradation of the reagents by light or other adverse conditions.

The kits may optionally include instructional materials containing directions (i.e., protocols) providing for the use of the reagents in the diagnosis, detection, and/or treatment of fibrosis (i.e., for example, pulmonary fibrosis) within a mammal. In particular the disease can include any one or more of the disorders described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Experimental

### Example I

### Animals And Bleomycin Administration

Wild type C57BL/6 mice used were purchased from Charles River Breeding Laboratories. Experiments comparing LPA₁^{-/-} and wild type mice used sex- and age or weight matched groups of LPA₁^{-/-} and littermate wild type mice produced by matings between mice heterozygous for the LPA₁ mutant allele. Contos et al., “Requirement for the IpA1 lysophosphatidic acid receptor gene in normal suckling behavior” Proc Natl Acad Sci USA 97:13384-13389 (2000). These LPA₁^{-/-} and littermate wild type mice were hybrids of the C57BL/6 and 129Sv/J genetic backgrounds. C57BL/6 mice received 0.05 or 0.075 units of bleomycin (Gensia Sicor Pharmaceuticals) in 50 or 75 μl sterile saline by intratracheal injection as indicated. LPA₁^{-/-} and littermate wild type mice received 2 or 3 units/kg of bleomycin in 50 μl sterile saline by intratracheal injection as indicated. All experiments were performed in accordance with National Institute of Health guidelines and protocols approved by the Massachusetts General Hospital Subcommittee on Research Animal Care.

### Example II

### Bronchoalveolar Lavage Recovery And Fractionation

Bronchoalveolar lavage (BAL) samples were obtained by lung lavage using two 0.5 ml aliquots of PBS. These samples were then provided for analysis of: i) chemoattractant activity, ii) LPA; iii) total protein; and iv) D-dimer concentrations. These BAL samples were centrifuged at 3000×g for 20 min at 4° C., and the supernatants transferred to siliconized low-binding eppendorf tubes (PGC Scientifics) for subsequent analysis.

However, BAL samples for analysis of leukocyte recruitment was performed with minor modifications. Lungs were lavaged with six 0.5 ml aliquots of PBS BAL fluid. These samples were centrifuged at 540×g at 4° C., and the pelleted cells were resuspended for cytospin and cytofluorimetric analysis.

Fractionation of BAL samples obtained for analysis of chemoattractant activity was performed as follows. Size exclusion centrifugation of BAL samples was performed with Microcon® Centrifugal Filter Units (Millipore) with molecular weight exclusion sizes of 30, 50 and 100 kDa, according to the manufacturer's instructions.

Heparin affinity chromatography was performed by loading BAL onto a 5 ml HiTrap Heparin HP column (Amersham Biosciences) equilibrated in 50 mM sodium acetare, pH 4.5, and elution with a linear gradient of 0 to 2M NaCl in 50 mM sodium acetate, pH 4.5, using an ÄKTA™ FPLC system (Amersham Biosciences).

Hydrophobic interaction chromatography was performed by dialyzing BAL samples against 50 mM sodium phosphate buffer, 1.7 M ammonium sulfate, pH 7.0, and then loading onto a 1 ml RESOURCE PHE column (Amersham Biosciences) equilibrated in the same buffer, and eluting with a linear gradient of 1.7 to 0.0 M ammonium sulfate.

Prior to use in chemotaxis assays, heparin binding affinity and surface hydrophobicity fractions were washed with PBS and returned to their original volumes using Centricon® Centrifugal Filter Units with molecular weight exclusion size of 3 kDa. Proteins in size exclusion centrifugation, heparin binding affinity, and surface hydrophobicity fractions were visualized by SDS-PAGE (4-20% Tris-HCI gels, Bio-Rad) and Comassie staining.

### Example III

### Isolation Of Primary Lung Fibroblasts

Lungs from unchallenged C57BL/6, LPA₁^{-/-} or littermate wild type mice were digested for 45 minutes at 37° C. in DMEM with 0.14 U/ml Liberase Blenzyme 3 (Roche), and 60 μg/ml DNAse 1 (Sigma), passed through a 70 μm filter, centrifuged at 540×g at 4° C. and plated in tissue culture flasks in DMEM with 15% (vol/vol) fetal bovine serum (FBS, Sigma). Cells were passaged when subconfluent following harvest with trypsin-EDTA (Cellgro®). To ensure that cultures contained almost purely fibroblasts, cells were used for experiments at passage 3 or higher.

### Example IV

### Fibroblast Chemotaxis Assays

After being serum-starved in DMEM without FBS overnight, subconfluent primary lung fibroblasts were harvested with non-enzymatic Cell Dissociation Solution (Sigma) to avoid possible proteolysis of surface chemoattractant receptors by trypsin. Harvested cells, at 5.0×10⁵ cells/ml in 50 μl of DMEM, were placed in the top of a 48-well modified Boyden microchemotaxis chamber (NeuroProbe). Duplicate 30 μl aliquots of either: i) 18:1 LPA (Avanti Polar Lipids) or PDGF-BB (R&D Systems) in DMEM with 0.1% fatty acid-free bovine serum albumin (Sigma); or ii) BAL, or BAL fractions, diluted 1:4 in DMEM, were placed in the bottom wells of the chamber and separated from the cells by a 8-μm-pore, polyvinylpyrrolidone-free polycarbonate filter (Osmonics) that had been coated with fibronectin (Sigma). The apparatus was incubated at 37° C. and 5% CO₂ for 3 hours, and cells migrating across the filter and adhering to the bottom side of the filter were stained with a commercially modified Wright's stain (Hema 3 stain set, Fisher Diagnostics) and counted, after cells remaining on the top side of the filter had been scraped off. Data from these experiments is presented as chemotactic indices, i.e. the number of cells migrating in response to BAL divided by the number of cells migrating to media controls.

### Example V

### BAL Sample LPA Analysis

BAL samples designated for LPA analysis were centrifuged at 3000×g for 20 min at 4° C., and supernatants were transferred to siliconized low binding microfuge tubes to prevent phospholipid binding. BAL total LPA concentrations were determined by an investigator blinded to the identity of the samples using electrospray ionization mass spectrometry (ESI-MS) according to previously published protocols. Xiao et al., “Electrospray ionization mass spectrometry analysis of lysophospholipids in human ascitic fluids: comparison of the lysophospholipid contents in malignant vs nonmalignant ascitic fluids” Anal Biochem 290:302-313 (2001).

### Example VI

### RNA Analysis

Ribonucleic acid (RNA) analysis of LPA receptor expression was performed using: i) mouse primary lung fibroblasts obtained in accordance with Example III; ii) mouse BAL leukocytes obtained in accordance with Example ; iii) mouse C166 yolk-sac-derived endothelial cell line cells (American Type Culture Collection): and iv) primary mouse cardiac endothelial cells.

Primary cardiac endothelial cells were isolated from heart tissue of C57BL/6 mice by sequential selection with anti-PECAM-1 and anti-ICAM-2 mAb-coated magnetic beads according to an established protocol. Allport et al., “Neutrophils from MMP-9- or neutrophil elastase-deficient mice show no defect in transendothelial migration under flow in vitro” J Leukoc Biol 71:821-828 (2002). Total cellular RNA was isolated using TRIzol® reagent (Invitrogen) according to the manufacturer's instructions. Quantitative real-time polymerase chain reaction (QPCR) analysis was performed using an Mx4000™ Multiplex Quantitative PCR System (Stratagene) as previously described. Means et al., “The Toll-like receptor 5 stimulus bacterial flagellin induces maturation and chemokine production in human dendritic cells” Journal of Immunology 170:5165-5175 (2003). Oligonucleotide primers for the genes investigated were designed using Primer Express software 1.0 (Applied Biosystems) and synthesized by Invitrogen.

### Example VII

### Histopathologic And Immunohistochemical Examination

Lungs excised for histopathology were fixed with 10% buffered formalin. Multiple paraffin embedded 5 μm sections of the entire mouse lung were stained with hematoxylin-eosin or Masson's trichrome. Sections for FSP1 immunostaining were treated with xylene for deparaffinization and rehydrated through a graded series of ethanols. Sections were then pretreated with 0.01% trypsin in PBS for 10 min and incubated with 1% BSA for 20 min. Sections were treated with primary rabbit biotinylated polyclonal antibody to FSP 148 for 2 hours, and stained by an indirect avidin-biotin immunoperoxidase method (Vectostain ABC kit, Vector Laboratories). Quantitative analysis of FSP1 staining was performed using IPLab image analysis software (Scanalytics), with the area of FSP1 staining presented as the percentage of a lung high power field HPF staining positive. This percentage was calculated by determining the area of positive FSP1 staining for 10 randomly selected non-overlapping HPFs for each lung section.

### Example VIII

### Hydroxyproline Assay

Lungs were homogenized in 1 ml of PBS, and a 0.5 ml aliquot was hydrolyzed in 6 N HCl at 120° C. for 12 h. A 25 μl aliquot was then desiccated, resuspended in 25 μl H₂0, and added to 0.5 ml of 1.4% chloramine T (Sigma), 10% n-propanol, and 0.5 M sodium acetate, pH 6.0. After 20 min of incubation at room temperature, 1 ml of Erlich's solution (1 M p-dimethylaminobenzaldehyde (Sigma) in 70% n-propanol, 20% perchloric acid) was added and a 15 min incubation at 65° C. was performed. Absorbance was measured at 550 nm and the amount of hydroxyproline was determined against a standard curve.

### Example IX

### Fibroblast Proliferation

Subconfluent passage 2 to 3 primary lung fibroblasts were seeded into 24 well plates (4×10⁴ fibroblasts/well) in DMEM with 15% FBS. After 8 hours in culture, cells were serum-starved in DMEM without FBS overnight. 800 μl aliquots of PDGF-BB, or BAL diluted 1:4 in serum-free DMEM, were then added to the cells for a total of 32 hours. Proliferation was determined by incorporation of [methyl-³H]thymidine (DuPont-NEN) during the final 8 hours of culture.

### Example X

### Evans Blue Dye Extravasation Assay

Evans blue dye extravasation was assessed in unchallenged and bleomycin-challenged mice using a modified technique. Reutershan et al., “Critical role of endothelial CXCR2 in LPS-induced neutrophil migration into the lung” J Clin Invest 116:695-702 (2006). Briefly, Evans blue dye (20 mg/kg; Sigma), was injected intravenously into mice 3 hours prior to sacrifice. At the time of sacrifice, blood was recovered into a heparinized syringe by cardiac puncture. The right ventricle was then perfused with PBS to remove intravascular dye from the lungs, which were then removed and homogenized. Evans blue was extracted from the homogenates by the addition of two volumes of formamide followed by incubation overnight at 60° C., followed by centrifugation at 5000×g for 30 min. The absorption of Evans blue in the lung supernatants and the plasma was measured at 620 nm and corrected for the presence of heme pigments as follows: A₆₂₀ (corrected)=A₆₂₀−(1.426×A₇₄₀+0.030). The concentrations of Evans blue in the lung and plasma were then determined against a standard curve, and the degree of vascular leak in each lung set presented as an Evans blue index, calculated as the ratio of the total amount of Evans blue in those lungs to the concentration of Evans blue in the plasma of that mouse.

### Example XI

### BAL Sample Total Protein And D-dimer

Total protein concentrations in BAL samples were determined using bicinchoninic acid (BCA) for colorimetric detection of protein, using a commercially available BCA™ Protein Assay Kit (Pierce) according to the manufacturer's instructions. Fibrin turnover in the airspaces was assessed by determining BAL concentrations of D-dimers, using a commercially available ELISA kit (Asserachrom D-Di, Diagnostica Stago), according to the manufacturer's instructions.

### Example XII

### BAL Sample Leukocyte Counts And Cytofluorimetry

Leukocytes from BAL samples were obtained as described above. Recovered live cells were counted by trypan blue exclusion using a hemocytometer. Percentages of macrophages and neutrophils were determined on preparations of cells centrifuged with a Cytospin 3 (Shandon) and stained with Hema 3 stain. Cells recovered from BAL samples were then incubated at 4° C. for 20 min with 2.4G2 anti-FcγIII/II receptor (BD Pharmingen), and stained with APC-conjugated anti-mouse CD3, PE conjugated anti-mouse CD4 and FITC-conjugated anti-mouse CD8 monoclonal antibodies (BD Pharmingen) at 4° C. for 30 minutes. Cytofluorimetry was performed using a FACS Caliber Cytometer (Becton-Dickinson) and analyzed using CellQuest software.

### Example XIII

### Human BAL Samples And Human BAL Fibroblasts

BAL samples were recovered from IPF patients and normal controls following instillation of sterile 0.9% saline by flexible fiberoptic bronchoscopy, performed both at the Instituto Nacional de Enfermedades Respiratorias and the Massachusetts General Hospital (MGH), as previously described. Selman et al., “Accelerated variant of idiopathic pulmonary fibrosis: clinical behavior and gene expression pattern” PLoS ONE 2 e482 (2007); and Medoff et al., “BLT1-mediated T cell trafficking is critical for rejection and obliterative bronchiolitis after lung transplantation” J Exp Med 202:97-110 (2005). Supernatants were kept at −70° C. until use. Pelleted cells recovered from BAL samples performed at the MGH were plated in DMEM with 20% (vol/vol) FBS and incubated at 37° C. in 5% CO₂. Cells growing at passage 3 or higher were analyzed as primary human BAL fibroblasts.

### Example IVX

### Statistical Analysis

Differences in fibroblast chemotatic and proliferative indices, LPA, total protein, D-dimer, FSP1 staining areas, Evans blue indices, and leukocyte counts between wild type and LPA₁^{-/-} mice or fibroblasts were tested for statistical significance by Student's t test using Microsoft® Excel software. Effects of genotype and bleomycin challenge on lung hydroxyproline were tested for statistically significant interaction by two-way analysis of variance for independent samples using VassarStats: Website for Statistical Computations. (faculty.vassar.edu/lowry/VassarStats.html). Differences in survival between wild type and LPA₁^{-/-} mice were tested for statistical significance by log rank test. P<0.05 was considered significant in all comparisons.

We claim:

1. A method, comprising:
a) providing:
i) a subject at risk for an injury, wherein said injury is likely to result in a fibrosis;
ii) a composition comprising an inhibitory compound having affinity for at least a fragment of a lysophosphatidic acid receptor;
b) administering said composition to said subject before said injury, under conditions such that said fibrosis is prevented or reduced.

2. The method of claim 1, wherein said injury comprises a pulmonary injury.

3. The method of claim 1, wherein said pulmonary injury is selected from the group consisting of toxin inhalation injury, surgical procedure injury, infection, and accidental injury.

4. The method of claim 1, wherein said fibrosis comprises symptoms selected from the group consisting of fibroblast migration and vascular leak.

5. The method of claim 1, wherein said composition further comprises at least one additional drug.

6. The method of claim 5, wherein said drug is selected from the group consisting of antiproliferative drugs, anticoagulant drugs, antithrombotic drugs, and antiplatelet drugs.

7. The method of claim 1, wherein said administering is selected from the group consisting of topical, oral, parenteral, pulmonary, anal, vaginal, ocular, and intranasal.

8. A method, comprising:
a) providing:
i) a subject comprising an injury, wherein said injury resulted in a fibrosis;
ii) a composition comprising an inhibitory compound having affinity for at least a fragment of a lysophosphatidic acid receptor;
b) administering said composition to said subject after said injury, under conditions such that said fibrosis is reduced.

9. The method of claim 8, wherein said injury comprises a pulmonary injury.

10. The method of claim 8, wherein said pulmonary injury is selected from the group consisting of toxin inhalation injury, surgical procedure injury, infection, and accidental injury.

11. The method of claim 8, wherein said fibrosis comprises symptoms selected from the group consisting of fibroblast migration and vascular leak.

12. The method of claim 8, wherein said composition further comprises at least one additional drug.

13. The method of claim 12, wherein said drug is selected from the group consisting of antiproliferative drugs, anticoagulant drugs, antithrombotic drugs, and antiplatelet drugs.

14. The method of claim 12, wherein said administering is selected from the group consisting of topical, oral, parenteral, pulmonary, anal, vaginal, ocular, and intranasal.

15. A method, comprising:
a) providing;
i) an isolated lysophosphatidic acid receptor, wherein said receptor is derived from a fibroblast; and
ii) a test compound capable of an interaction with said receptor;
b) contacting said receptor with said test compound; and
c) detecting said interaction of said receptor with said test compound.

16. The method of claim 15, wherein said fibroblast is derived from a pulmonary tissue.

17. The method of claim 15, wherein said test compound comprises a protein.

18. The method of claim 15, wherein said test compound comprises a small organic molecule.

19. The method of claim 17, wherein said protein comprises a fusion peptide.

20. The method of claim 15, wherein said test compound comprises a nucleic acid.

21. The method of claim 17, wherein said protein comprises an antibody.

22. The method of claim 17, wherein said protein comprises a peptide.

23. A method, comprising:
a) providing:
i) a subject comprising a progressive injury, wherein said injury promotes fibrosis;
ii) a composition comprising an inhibitory compound having affinity for at least a fragment of a lysophosphatidic acid receptor; and
b) administering said composition to said subject before said injury, under conditions such that said fibrosis is prevented or reduced.

24. The method of claim 23, wherein said injury comprises a pulmonary injury.

25. The method of claim 23, wherein said progressive injury results in an increase in said fibrosis.

26. The method of claim 24, wherein said pulmonary injury is selected from the group consisting of toxin inhalation injury, surgical procedure injury, infection, and accidental injury.

27. The method of claim 23, wherein said fibrosis comprises symptoms selected from the group consisting of fibroblast migration and vascular leak.

28. The method of claim 23, wherein said composition further comprises at least one additional drug.

29. The method of claim 28, wherein said drug is selected from the group consisting of antiproliferative drugs, anticoagulant drugs, antithrombotic drugs, and antiplatelet drugs.

30. The method of claim 23, wherein said administering is selected from the group consisting of topical, oral, parenteral, pulmonary, anal, vaginal, ocular, and intranasal.

31. A kit comprising:
a) a nucleic acid capable of hybridizing to at least a portion of an LPA₁ receptor deoxyribonucleic acid (DNA) sequence;
b) at least one sample comprising said LPA₁ receptor DNA sequence; and
c) a set of instructions for using said nucleic acid to detect said LPA₁ receptor DNA sequence.

32. The kit of claim 31, wherein said at least one sample comprises a patient sample.

33. The kit of claim 32, wherein said patient sample comprises lung tissue.

34. The kit of claim 31, wherein said at least one sample comprises a wild-type fibroblast cell culture sample.

35. The kit of claim 31, wherein said DNA sequence comprises an LPA₁ coding region.

36. The kit of claim 31, wherein said nucleic acid comprises a primer.

37. The kit of claim 31, wherein said kit further comprises at least one polymerase enzyme.

38. The kit of claim 31, wherein said instructions further provide for using said DNA sequence detection to diagnose a fibrosis.

39. The kit of claim 38, wherein said fibrosis is pulmonary fibrosis.

40. The kit of claim 31, wherein said instructions further diagnose fibrosis by comparing said patient sample detected DNA sequence to said cell culture detected DNA sequence.

41. A kit comprising:
a) a nucleic acid capable of hybridizing to at least a portion of an LPA₁ receptor messenger ribonucleic acid (mRNA) sequence;
b) at least one sample comprising said LPA, receptor mRNA sequence; and
c) a set of instructions for using said nucleic acid to detect said LP/¹i_{’} receptor mRNA sequence.

42. The kit of claim 41, wherein said nucleic acid sequence comprises a primer.

43. The kit of claim 41, wherein said kit further comprises at least one polymerase.

44. The kit of claim 41, wherein said at least one sample comprises a patient sample.

45. The kit of claim 44, wherein said patient sample comprises lung tissue.

46. The kit of claim 41, wherein said at least one sample comprises a wild-type fibroblast cell culture sample.

47. The kit of claim 41, wherein said mRNA sequence comprises an LPA₁ coding region.

48. The kit of claim 41, wherein said instructions further provide for using said mRNA sequence detection to diagnose fibrosis.

49. The kit of claim 48, wherein said fibrosis is pulmonary fibrosis.

50. The kit of claim 48, wherein said instructions further diagnose fibrosis by comparing said patient sample detected mRNA sequence to said cell culture detected mRNA sequence.

51. A kit comprising:
a) at least one antibody capable of binding to an LPA₁ receptor protein;
b) at least one sample comprising said LPA₁ receptor protein; and
c) a set of instructions for using said at least one antibody to detect said LPA₁ receptor protein.

52. The kit of claim 51, wherein said at least one antibody comprises a first labeled antibody.

53. The kit of claim 51, wherein said at least one antibody comprises a second labeled antibody.

54. The kit of claim 51, wherein said at least one sample comprises a patient sample.

55. The kit of claim 54, wherein said patient sample comprises lung tissue.

56. The kit of claim 51, wherein said at least one sample comprises a wild-type fibroblast cell culture sample.

57. The kit of claim 52, wherein said first antibody comprises a high affinity for an LPA₁ receptor epitope.

58. The kit of claim 53, wherein said second antibody comprises a high affinity for said first antibody.

59. The kit of claim 51, wherein said instructions further provide for using said LPA₁ receptor protein detection to diagnose fibrosis.

60. The kit of claim 59, wherein said fibrosis is pulmonary fibrosis.

61. The kit of claim 59, wherein said instructions further diagnose fibrosis by comparing said patient sample detected LPA₁ receptor protein to said cell culture detected LPA₁ receptor protein.

62. A kit comprising:
a) an LPA₁ receptor inhibitor; and
b) a pharmaceutically acceptable carrier capable of administering said inhibitor to a subject.

63. The kit of claim 62, wherein said inhibitor comprises a nucleic acid capable of hybridizing to at least a portion of an LPA₁ receptor coding region.

64. The kit of claim 62, wherein said inhibitor comprises an antibody capable of binding to an LPA₁ receptor protein.

65. The kit of claim 62, wherein said inhibitor comprises a small organic molecule capable of binding to an LPA₁ receptor protein.

66. The kit of claim 62, wherein said inhibitor comprises a protein capable of binding to an LPA₁ receptor protein.

67. The kit of claim 62, wherein said kit further comprises a set of instructions for administering said receptor inhibitor to said subject.


SEQUENCE LISTING (8 sequences)

SEQ ID NO: 1 - DNA, 3182 bp, Homo sapiens
acggcgcgctgggctcacactgtcccgccgcggacgggctttgtggttgggggcgcgcgtgcgagtgccagtgagagtgtgggtgcgcgctgtgggccgcggcgcgggtgggtggccgtgcgttcttgcgagccggcctgcaggaggcgaggctcccctggcctcccgcacccagcggcggaccgagcccctggagggaagttgccgcagccgcccgggccgccggccctcctgtcccgcgccaggtggacgtctgatttatgaagctccccatccacctatctgagtacctgacttctcaggactgacacctacagcatcaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaggctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccatttagactacactaactagacttttaaaagattttgtgtggtttggtgcaagtcagaataaattctggctagttgaatccacaacttcatttatatacaggcttcccttttttatttttaaaggatacgtttcacttaataaacacgtttatgcctatcagcatgtttgtgatggatgagactatggactgcttttaaactaccataattccattttttcccttacataggaaaactgtaagttggaattatcttttgtttagaaagcatgcatgtaatgtatgtatgcagtatgccttacttaaaaagattaaaaggatactaatgttaaatcttctaggaaatagaacctagacttcaaagccagtatttgtttaggtcatgaagcaaacaatgctctaatcacaatattaactgtttaattaaaatgttgtaacaagtataaaacagggaatgtaagtttattaccaaagtgatatgtattccaaaaaagtcatagaagatgaagcactataatattgttcccatatatttaaaatacccaagtacattctaattaccagtatatcagaggaaaattttcgtagtctttgtaaaataatatactcatcatagaaaacttgaaaaatgcagaaatgtataaaaaagcaaaaatgattactgataatatcacaacccagaagtaaccacctttaaaaagcaacccccatgtatgcctatatgtgtattgtatactttttttacataattggagtcatactgtaaacagttttataagtagatctttttcattgcaaaattgccacattttcttatggcattaaaaattttacaaaaacataattttaatggctatattatattccatttaatggatgcaactcagtttatttaaccattcccatgttgttaactatttaggttgtttctaattttcattattataaagttgcagaaatttggtgtacataaaactgtctccatataattgattattaggatatattcccatgaaggattctttttttaaaaaaatgtgaaatgtcatcttgtacttacacctttcatgaaaagggatttcctgcttttgtactgcatgggtggcagttgtgaggaaaagccagtcaaatgacctttttacaaaagaaatgcagtggtcacttcagttgagagtgactttttaatacaacaagatcaactagaagaattcaactgtctcaagaatcaaggtaccccaatatatctcgcaattccaaactttgtttgagggactcgttatccagctcttggtagccacacctgcaatgtaaaatggaagaaaatgcaaagaaaccaaatgtgccgagtgaataaaggattgtcatatcaaaaaaaaaaaaaaaa

SEQ ID NO: 2 - protein, 364 aa, Homo sapiens
MAAISTSIPVISQPQFTAMNEPQCFYNESIAFFYNRSGKHLATEWNTVSKLVMGLGITVCIFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIENCSNMAPLYSDSYLVFWAIFNLVTFVVMVVLYAHIFGYVRQRTMRMSRHSSGPRRNRDTMMSLLKTVVIVLGAFIICWTPGLVLLLLDVCCPQCDVLAYEKFFLLLAEFNSAMNPIIYSYRDKEMSATFRQILCCQRSENPTGPTEGSDRSASSLNHTILAGVHSNDHSVV

SEQ ID NO: 3 - DNA, 3104 bp, Homo sapiens
acggcgcgctgggctcacactgtcccgccgcggacgggctttgtggttgggggcgcgcgtgcgagtgccagtgagagtgtgggtgcgcgctgtgggccgcggcgcgggtgggtggccgtgcgttcttgcgagccggcctgcaggaggcgaggctcccctggcctcccgcacccagcggcggaccgagcccctggagggaagttgccgcagccgcccgggccgccggccctcctgtcccgcgccaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaggctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccatttagactacactaactagacttttaaaagattttgtgtggtttggtgcaagtcagaataaattctggctagttgaatccacaacttcatttatatacaggcttcccttttttatttttaaaggatacgtttcacttaataaacacgtttatgcctatcagcatgtttgtgatggatgagactatggactgcttttaaactaccataattccattttttcccttacataggaaaactgtaagttggaattatcttttgtttagaaagcatgcatgtaatgtatgtatgcagtatgccttacttaaaaagattaaaaggatactaatgttaaatcttctaggaaatagaacctagacttcaaagccagtatttgtttaggtcatgaagcaaacaatgctctaatcacaatattaactgtttaattaaaatgttgtaacaagtataaaacagggaatgtaagtttattaccaaagtgatatgtattccaaaaaagtcatagaagatgaagcactataatattgttcccatatatttaaaatacccaagtacattctaattaccagtatatcagaggaaaattttcgtagtctttgtaaaataatatactcatcatagaaaacttgaaaaatgcagaaatgtataaaaaagcaaaaatgattactgataatatcacaacccagaagtaaccacctttaaaaagcaacccccatgtatgcctatatgtgtattgtatactttttttacataattggagtcatactgtaaacagttttataagtagatctttttcattgcaaaattgccacattttcttatggcattaaaaattttacaaaaacataattttaatggctatattatattccatttaatggatgcaactcagtttatttaaccattcccatgttgttaactatttaggttgtttctaattttcattattataaagttgcagaaatttggtgtacataaaactgtctccatataattgattattaggatatattcccatgaaggattctttttttaaaaaaatgtgaaatgtcatcttgtacttacacctttcatgaaaagggatttcctgcttttgtactgcatgggtggcagttgtgaggaaaagccagtcaaatgacctttttacaaaagaaatgcagtggtcacttcagttgagagtgactttttaatacaacaagatcaactagaagaattcaactgtctcaagaatcaaggtaccccaatatatctcgcaattccaaactttgtttgagggactcgttatccagctcttggtagccacacctgcaatgtaaaatggaagaaaatgcaaagaaaccaaatgtgccgagtgaataaaggattgtcatatcaaaaaaaaaaaaaaaa

SEQ ID NO: 4 - protein, 364 aa, Homo sapiens; identical to SEQ ID NO: 2

SEQ ID NO: 5 - DNA, 1148 bp, Homo sapiens
cttctttaaatttctttctaggatgttcacttcttctccacaatgaatgagtgtcactatgacaagcacatggactttttttataataggagcaacactgatactgtcgatgactggacaggaacaaagcttgtgattgttttgtgtgttgggacgtttttctgcctgtttatttttttttctaattctctggtcatcgcggcagtgatcaaaaacagaaaatttcatttccccttctactacctgttggctaatttagctgctgccgatttcttcgctggaattgcctatgtattcctgatgtttaacacaggcccagtttcaaaaactttgactgtcaaccgctggtttctccgtcaggggcttctggacagtagcttgactgcttccctcaccaacttgctggttatcgccgtggagaggcacatgtcaatcatgaggatgcgggtccatagcaacctgaccaaaaagagggtgacactgctcattttgcttgtctgggccatcgccatttttatgggggcggtccccacactgggctggaattgcctctgcaacatctctgcctgctcttccctggcccccatttacagcaggagttaccttgttttctggacagtgtccaacctcatggccttcctcatcatggttgtggtgtacctgcggatctacgtgtacgtcaagaggaaaaccaacgtcttgtctccgcatacaagtgggtccatcagccgccggaggacacccatgaagctaatgaagacggtgatgactgtcttaggggcgtttgtggtatgctggaccccgggcctggtggttctgctcctcgacggcctgaactgcaggcagtgtggcgtgcagcatgtgaaaaggtggttcctgctgctggcgctgctcaactccgtcgtgaaccccatcatctactcctacaaggacgaggacatgtatggcaccatgaagaagatgatctgctgcttctctcaggagaacccagagaggcgtccctctcgcatcccctccacagtcctcagcaggagtgacacaggcagccagtacatagaggatagtattagccaaggtgcagtctgcaataaaagcacttcctaaactctggatgcctctcggcccacccaggtgatgactgtcttagg

SEQ ID NO: 6 - protein, 353 aa, Homo sapiens
MNECHYDKHMDFFYNRSNTDTVDDWTGTKLVIVLCVGTFFCLFIFFSNSLVIAAVIKNRKFHFPFYYLLANLAAADFFAGIAYVFLMFNTGPVSKTLTVNRWFLRQGLLDSSLTASLTNLLVIAVERHMSIMRMRVHSNLTKKRVTLLILLVWAIAIFMGAVPTLGWNCLCNISACSSLAPIYSRSYLVFWTVSNLMAFLIMVVVYLRIYVYVKRKTNVLSPHTSGSISRRRTPMKLMKTVMTVLGAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQGAVCNKSTS

SEQ ID NO: 7 - DNA, 3650 bp, Mus musculus
agcacagtgctgccctccgtaggctccgggttgtgctgggtgaggcttgggttgggttggcccggcggctgcgtgaactgcggagctggacctagcaggcttacagttcctcctagcatgaccgagatctgatcagccaacccgcgcattgctttttgtgcctggcactgcagtgcagggggcctcttcatcgccccaaactacagcactgtcatggcagctgcctctacttccagccctgtaatttcacagccccagttcacagccatgaacgaacaacagtgcttctacaatgagtctatcgccttcttttataaccggagtgggaaatatctagccacagaatggaacacagtgagcaagctggtgatgggactgggcatcactgtttgcgtgttcatcatgttggccaatctcctggtcatggtggcaatctacgtcaaccgccgcttccatttccctatttattacttgatggccaacctggctgctgcagacttcttcgctggattggcctacttctacctgatgttcaatacaggacctaatacccggagactgactgttaacacgtggctcctccggcagggcctcattgacaccagcctgacagcttctgtggccaacctgctggctattgctatcgagaggcacatcacggttttccgcatgcagctccatacacgaatgagcaaccggcgcgtggtggtggtgattgtagtcatctggactatggccattgtgatgggtgctatgcccactgtgggctggaactgcatctgtgatatcgatcactgttccaacatggcacccctctacagtgactcctacttagtcttctgggccattttcaacctggtgacctttgtggtcatggtggttctctacgctcacatctttggctatgttcgccagaggactatgaggatgtctcggcatagttctggacccaggaggaatcgggacaccatgatgagccttctgaagactgtggtcattgtgcttggtgcctttattgtctgctggactccgggattggtcttgttattgctggatgtgtgctgcccgcagtgcgatgtcctggcctatgagaagttcttcctcctcctggccgagttcaactctgctatgaaccccatcatctactcctaccgcgacaaagagatgagcgccaccttcaggcagatcctgtgttgccagcgcaacgagaaccctaatggccccacggaaggctctgaccgctctgcctcctccctcaaccacaccattctggctggagttcacagcaacgaccactctgtggtttagaaggaagccagccggcctctgtggatctgtgaaccccaccctaccccccattgccagggcaaggtggggagccagaggagatgaggacactcctgtacttaacactaaccaatggcagtatttgtccctagacccaagagacttgaggatgaatttatttggcaggccccatcttctcctttggaaaacagaaggggaccgtcttgtggtggaattgagaaatggactctggggtgaccgtgtagcattcactaactagacttaaaagattttatgtggtttggcttaagccaggaaaaaaaaatctgctgaattgagtatacaatcgagtatacacaggcttcccctttaaagaacaaacaatacattgcatttattaatgagtatgtttatgcctgacagcatgtttgtgatcgaaaagactgctaaactgacatagatgagttgtttttttttttttgttttttgtttttttttacatgatggaggaaaagtataaattagaatgatttttgtgtttgtttagaaagcaagcatgtggtgtgtgtattcagtatgcctttctttaaagataaaaggccactattttaaatcttctagggaatagaagaatctagtaaaaaccagtattcatttaggctacaggaaaaaccatatcctaatcaattaccttttaattaaagtaatgaaatatacatgaaaggcaaagtaatgtgagcttgtcacccaaagagtgtgtgctctccaaacgctggaggagatgaagctgtagcgttgtccctgcatagtgaagatacccacgtgcgttctcagtgccagaccctcagtgggacttgttttaaagcctgtggttttccaagttagaaaataatacctacttactatagaaaacttgaaaattgcagaactgtgtgaaaaaagaaaaaagaaagaaagattgctgatatacgacctggaagtagccatctttccctgctcacccacgtatgcctatagacatatattatatacctttttttttttttacataatcgggttgatattgtaaaatgttttgtaactttttctttcagaattgccaggtttttctatggcattttttttcaaaaacgttagcggctatataatattccatttaatggatgcagctcagtttatttagtaccattcctgtattgttgactatgcttttggcttttttttcataatattggaagaatcttgtgtatataaaactttgccagtatcattgatgatttacttgggctccattcccatgaaggacctacttttaaaatgtgaaatgtcctcatgtgttgtcactttataaaaagggatattctaccctcaaactgcaggggtgaccatgctgacaaaaagctcagaaattacctttttacaaaagaaacacagtggctactttagttgcgaatgggttcttgacaagatgtttcccaataacccagacccttaacataactagcaaatttactttagtaggaactggtgatccctttctggacacaagaaaacacaaagacatcaagcatcagagtgaatccagcaatgcagtaccaagcagcctgggtggggtgtgctagcaactggctgctgtgtggtctccagcaatcacccaatggacaccctgtgttcagagatgcccagtacagtgtcctggagactaaggtctctaatgtcatgctatgaggaacaaggaggaagaagaatagtcctgaagcaggtgtggagaggtcaggaggacgtctgagagaaggcagtcctgccaagagtggcattctagggaggaggaaggcagggtgtggcttgtgtctggacagtcagtggtaggcatgtgacatttgcctggtggaaaaaaaataagtaggcggagatccagttagcaggacaaagattttgctcgaggattcccccaatccaagaaatttaaactggaaatgagtgaaccgaacttggacttttattgattccccttttatgggggaggtaaggactatttgaattgaaaagcatactaattgaaccttaataaatcattctcaatcagtgtttggcgatgtgtggttctggtctgtgctttttttctattttcgagaagcctctggctgtgctgtgatggcttctctctctgaccttctttaacagtctgaggcagcccaacaactgttcctttagctcagatactggttcctcatccatgagattcatgagagacgtgttacctcaatggaatgagtactagagcaaggtatttagagagattttttttattatttatttatttattttgaataaaatgtatgtaataaataagataaaataaa

SEQ ID NO: 8 - protein, 364 aa, Mus musculus
MAAASTSSPVISQPQFTAMNEQQCFYNESIAFFYNRSGKYLATEWNTVSKLVMGLGITVCVFIMLANLLVMVAIYVNRRFHFPIYYLMANLAAADFFAGLAYFYLMFNTGPNTRRLTVNTWLLRQGLIDTSLTASVANLLAIAIERHITVFRMQLHTRMSNRRVVVVIVVIWTMAIVMGAMPTVGWNCICDIDHCSNMAPLYSDSYLVFWAIFNLVTFVVMVVLYAHIFGYVRQRTMRMSRHSSGPRRNRDTMMSLLKTVVIVLGAFIVCWTPGLVLLLLDVCCPQCDVLAYEKFFLLLAEFNSAMNPIIYSYRDKEMSATFRQILCCQRNENPNGPTEGSDRSASSLNHTILAGVHSNDHSVV